SEQUENCE LISTING (97 sequences)

SEQ ID NO: 1 - protein, 165 aa, synthetic construct
MKVAKDLVVSLAYQVRTEDGVLVDESPVSAPLDYLHGHGSLISGLETALEGHEVGDKFDVAVGANDAYGQYDENLVQRVPKDVFMGVDELQVGMRFLAETDQGPVPVEITAVEDDHVVVDGNHMLAGQNLKFNVEVVAIREATEEELAHGHVHGAHDHHHDHDHD

SEQ ID NO: 2 - protein, 214 aa, synthetic construct
MAFKNDDQKSAYALGASLGRYMENSLKEQEKLGIKLDKDQLIAGVQDAFADKSKLSDQEIEQTLQAFEARVKSSAQAKMEKDAADNEAKGKEYREKFAKEKGVKTSSTGLVYQVVEAGKGEAPKDSDTVVVNYKGTLIDGKEFDNSYTRGEPLSFRLDGVIPGWTEGLKNIKKGGKIKLVIPPELAYGKAGVPGIPPNSTLVFDVELLDVKPAP

SEQ ID NO: 3 - protein, 149 aa, synthetic construct
MSESVQSNSAVLVHFTLKLDDGTTAESTRNNGKPALFRLGDASLSEGLEQHLLGLKVGDKTTFSLEPDAAFGVPSPDLIQYFSRREFMDAGEPEIGAIMLFTAMDGSEMPGVIREINGDSITVDFNHPLAGQTVHFDIEVLEIDPALEA

SEQ ID NO: 4 - protein, 164 aa, synthetic construct
KVAKDLVVSLAYQVRTEDGVLVDESPVSAPLDYLHGHGSLISGLETALEGHEVGDKFDVAVGANDAYGQYDENLVQRVPKDVFMGVDELQVGMRFLAETDQGPVPVEITAVEDDHVVVDGNHMLAGQNLKFNVEVVAIREATEEELAHGHVHGAHDHHHDHDHD

SEQ ID NO: 5 - protein, 213 aa, synthetic construct
AFKNDDQKSAYALGASLGRYMENSLKEQEKLGIKLDKDQLIAGVQDAFADKSKLSDQEIEQTLQAFEARVKSSAQAKMEKDAADNEAKGKEYREKFAKEKGVKTSSTGLVYQVVEAGKGEAPKDSDTVVVNYKGTLIDGKEFDNSYTRGEPLSFRLDGVIPGWTEGLKNIKKGGKIKLVIPPELAYGKAGVPGIPPNSTLVFDVELLDVKPAP

SEQ ID NO: 6 - protein, 148 aa, synthetic construct
SESVQSNSAVLVHFTLKLDDGTTAESTRNNGKPALFRLGDASLSEGLEQHLLGLKVGDKTTFSLEPDAAFGVPSPDLIQYFSRREFMDAGEPEIGAIMLFTAMDGSEMPGVIREINGDSITVDFNHPLAGQTVHFDIEVLEIDPALEA

SEQ ID NO: 7 - protein, 7 aa, synthetic construct
GGGSGGG

SEQ ID NO: 8 - protein, 11 aa, synthetic construct
GGGSGGGSGGG

SEQ ID NO: 9 - protein, 15 aa, synthetic construct
GGGSGGGSGGGSGGG

SEQ ID NO: 10 - protein, 19 aa, synthetic construct
GGGSGGGSGGGSGGGSGGG

SEQ ID NO: 11 - protein, 23 aa, synthetic construct
GGGSGGGSGGGSGGGSGGGSGGG

SEQ ID NO: 12 - protein, 43 aa, synthetic construct
GGGSGGGSGGGYRYRQGGGSGGGSGGGYRYRQGGGSGGGSGGG

SEQ ID NO: 13 - protein, 43 aa, synthetic construct
GGGSGGGSGGGRVRQRGGGSGGGSGGGRVRQRGGGSGGGSGGG

SEQ ID NO: 14 - protein, 55 aa, synthetic construct
GGGSGGGSGGGSGGGYRYRQGGGSGGGSGGGSGGGYRYRQGGGSGGGSGGGSGGG

SEQ ID NO: 15 - protein, 55 aa, synthetic construct
GGGSGGGSGGGSGGGRVRQRGGGSGGGSGGGSGGGRVRQRGGGSGGGSGGGSGGG

SEQ ID NO: 16 - protein, 59 aa, synthetic construct
GGGSGGGSGGGSGGGSGGGYRYRQGGGSGGGSGGGSGGGYRYRQGGGSGGGSGGGSGGG

SEQ ID NO: 17 - protein, 59 aa, synthetic construct
GGGSGGGSGGGSGGGSGGGRVRQRGGGSGGGSGGGSGGGRVRQRGGGSGGGSGGGSGGG

SEQ ID NO: 18 - protein, 547 aa, synthetic construct
MKVAKDLVVSLAYQVRTEDGVLVDESPVSAPLDYLHGHGSLISGLETALEGHEVGDKFDVAVGANDAYGQYDENLVQRVPKDVFMGVDELQVGMRFLAETDQGPVPVEITAVEDDHVVVDGNHMLAGQNLKFNVEVVAIREATEEELAHGHVHGAHDHHHDHDHDGGGSGGGSGGGSGGGSGGGSGGGKVAKDLVVSLAYQVRTEDGVLVDESPVSAPLDYLHGHGSLISGLETALEGHEVGDKFDVAVGANDAYGQYDENLVQRVPKDVFMGVDELQVGMRFLAETDQGPVPVEITAVEDDHVVVDGNHMLAGQNLKFNVEVVAIREATEEELAHGHVHGAHDHHHDHDHDGGGSGGGSGGGSGGGSGGGSGGGTLTVQARQLLSGIVQQQNNELRAIEAQQHLLQLTVWGTKQLQARELAVERYLKDQQLLGIWGASGKLIATTAVPWNASWSNKSLEQIWNNMTWMEWDREINEYTSLIYSLIEESQNQQEKNEQELLELDKWASLWNWFNITNWLWYHGHDHDHDGGGSYRYRQGGGHHHHHH

SEQ ID NO: 19 - protein, 599 aa, synthetic construct
MKVAKDLVVSLAYQVRTEDGVLVDESPVSAPLDYLHGHGSLISGLETALEGHEVGDKFDVAVGANDAYGQYDENLVQRVPKDVFMGVDELQVGMRFLAETDQGPVPVEITAVEDDHVVVDGNHMLAGQNLKFNVEVVAIREATEEELAHGHVHGAHDHHHDHDHDGGGSGGGSGGGSGGGYRYRQGGGSGGGSGGGSGGGYRYRQGGGSGGGSGGGSGGGKVAKDLVVSLAYQVRTEDGVLVDESPVSAPLDYLHGHGSLISGLETALEGHEVGDKFDVAVGANDAYGQYDENLVQRVPKDVFMGVDELQVGMRFLAETDQGPVPVEITAVEDDHVVVDGNHMLAGQNLKFNVEVVAIREATEEELAHGHVHGAHDHHHDHDHDGGGSGGGSGGGSGGGYRYRQGGGSGGGSGGGSGGGYRYRQGGGSGGGSGGGSGGGTLTVQARQLLSGIVQQQNNELRAIEAQQHLLQLTVWGTKQLQARELAVERYLKDQQLLGIWGASGKLIATTAVPWNASWSNKSLEQIWNNMTWMEWDREINEYTSLIYSLIEESQNQQEKNEQELLELDKWASLWNWFNITNWLWYHGHDHDHDHHHHHH

SEQ ID NO: 20 - protein, 5 aa, synthetic construct
YRYRQ

SEQ ID NO: 21 - protein, 5 aa, synthetic construct
RVRQR

SEQ ID NO: 22 - protein, 5 aa, synthetic construct
QRQRQ

SEQ ID NO: 23 - protein, 5 aa, synthetic construct
FRQRG

SEQ ID NO: 24 - protein, 5 aa, synthetic construct
FRQRQ

SEQ ID NO: 25 - protein, 5 aa, synthetic construct
RYRQR

SEQ ID NO: 26 - protein, 5 aa, synthetic construct
RQRQR

SEQ ID NO: 27 - protein, 5 aa, synthetic construct
YRQSR

SEQ ID NO: 28 - protein, 5 aa, synthetic construct
YKYRQ

SEQ ID NO: 29 - protein, 5 aa, synthetic construct
LRYRQ

SEQ ID NO: 30 - protein, 5 aa, synthetic construct
YRQRA

SEQ ID NO: 31 - protein, 5 aa, synthetic construct
VRYRQ

SEQ ID NO: 32 - protein, 5 aa, synthetic construct
QRQTR

SEQ ID NO: 33 - protein, 5 aa, synthetic construct
YRQTR

SEQ ID NO: 34 - protein, 5 aa, synthetic construct
PRYRQ

SEQ ID NO: 35 - protein, 5 aa, synthetic construct
RFSQR

SEQ ID NO: 36 - protein, 5 aa, synthetic construct
WQRQR

SEQ ID NO: 37 - protein, 5 aa, synthetic construct
QYRQR

SEQ ID NO: 38 - protein, 5 aa, synthetic construct
VRQRQ

SEQ ID NO: 39 - protein, 5 aa, synthetic construct
RYTQR

SEQ ID NO: 40 - protein, 5 aa, synthetic construct
AYRQR

SEQ ID NO: 41 - protein, 5 aa, synthetic construct
YQRQR

SEQ ID NO: 42 - protein, 5 aa, synthetic construct
RYSQR

SEQ ID NO: 43 - protein, 5 aa, synthetic construct
EWVAQ

SEQ ID NO: 44 - protein, 5 aa, synthetic construct
EWALQ

SEQ ID NO: 45 - protein, 5 aa, synthetic construct
DYFLQ

SEQ ID NO: 46 - protein, 5 aa, synthetic construct
DYALQ

SEQ ID NO: 47 - protein, 5 aa, synthetic construct
EYWLQ

SEQ ID NO: 48 - protein, 5 aa, synthetic construct
DWALQ

SEQ ID NO: 49 - protein, 5 aa, synthetic construct
DWYLQ

SEQ ID NO: 50 - protein, 5 aa, synthetic construct
DYWLQ

SEQ ID NO: 51 - protein, 5 aa, synthetic construct
EYVAQ

SEQ ID NO: 52 - protein, 5 aa, synthetic construct
DYVAQ

SEQ ID NO: 53 - protein, 5 aa, synthetic construct
DWVAQ

SEQ ID NO: 54 - protein, 5 aa, synthetic construct
EYVLQ

SEQ ID NO: 55 - protein, 5 aa, synthetic construct
EWIAQ

SEQ ID NO: 56 - protein, 5 aa, synthetic construct
WYALQ

SEQ ID NO: 57 - protein, 5 aa, synthetic construct
EYALQ

SEQ ID NO: 58 - protein, 5 aa, synthetic construct
EYFLQ

SEQ ID NO: 59 - protein, 5 aa, synthetic construct
YVLQG

SEQ ID NO: 60 - protein, 5 aa, synthetic construct
FVLQG

SEQ ID NO: 61 - protein, 5 aa, synthetic construct
YALQG

SEQ ID NO: 62 - protein, 5 aa, synthetic construct
FALQG

SEQ ID NO: 63 - protein, 9 aa, synthetic construct
APRYRQRAA

SEQ ID NO: 64 - protein, 9 aa, synthetic construct
GDYALQGPG

SEQ ID NO: 65 - protein, 262 aa, synthetic construct
MHKWFLRAAVVAAVGFGLPTLIATTAQAAAVAAPTPRAPLAPPLAEDRSYRTWRVEDYVEAWERYHGREMTEDERENLARGCIGVTVVNLNREDLSNPPLNLSFGSLRTAEAVQAALNKIVDTHPSPAQYEAAVAKDPILKRLKNVVKALPSWIDSAKLKASIFSKRFYSWQNPDWSEERAHTTYRPDRETDQVDMSTYRYRARPGYVNFDYGWFDQDTNTWWHANHEEPRMVVYQSTLRHYSRPLQDFDEQVFTVAFAKKD

SEQ ID NO: 66 - protein, 5 aa, synthetic construct
EAAAK

SEQ ID NO: 67 - protein, 10 aa, synthetic construct
EAAAKEAAAK

SEQ ID NO: 68 - protein, 15 aa, synthetic construct
EAAAKEAAAKEAAAK

SEQ ID NO: 69 - protein, 20 aa, synthetic construct
EAAAKEAAAKEAAAKEAAAK

SEQ ID NO: 70 - protein, 40 aa, synthetic construct
EAAAKEAAAKYRYRQEAAAKEAAAKYRYRQEAAAKEAAAK

SEQ ID NO: 71 - protein, 40 aa, synthetic construct
EAAAKEAAAKRVRQREAAAKEAAAKRVRQREAAAKEAAAK

SEQ ID NO: 72 - protein, 55 aa, synthetic construct
EAAAKEAAAKEAAAKYRYRQEAAAKEAAAKEAAAKYRYRQEAAAKEAAAKEAAAK

SEQ ID NO: 73 - protein, 55 aa, synthetic construct
EAAAKEAAAKEAAAKRVRQREAAAKEAAAKEAAAKRVRQREAAAKEAAAKEAAAK

SEQ ID NO: 74 - protein, 70 aa, synthetic construct
EAAAKEAAAKEAAAKEAAAKYRYRQEAAAKEAAAKEAAAKEAAAKYRYRQEAAAKEAAAKEAAAKEAAAK

SEQ ID NO: 75 - protein, 70 aa, synthetic construct
EAAAKEAAAKEAAAKEAAAKRVRQREAAAKEAAAKEAAAKEAAAKRVRQREAAAKEAAAKEAAAKEAAAK

SEQ ID NO: 76 - protein, 114 aa, synthetic construct
SPKRLSAAIGKAWEEGVCGIRSATRLENIMWKQISNELNHILLENDMKFTVVVGDVAGILAQGKKMIRPQPMEHKYSWKSWGKAKIIGADVQNTTFIIDGPNTPECPDDQRAWN

SEQ ID NO: 77 - protein, 151 aa, synthetic construct
EQYKFQADSPKRLSAAIGKAWEEGVCGIRSATRLENIMWKQISNELNHILLENDMKFTVVVGDVAGILAQGKKMIRPQPMEHKYSWKSWGKAKIIGADVQNTTFIIDGPNTPECPDDQRAWNIWEVEDYGFGIFTTNIWLKLRDSYTQVAD

SEQ ID NO: 78 - protein, 5 aa, synthetic construct
RYESK

SEQ ID NO: 79 - protein, 146 aa, synthetic construct
TLTVQARQLLSGIVQQQNNELRAIEAQQHLLQLTVWGTKQLQARELAVERYLKDQQLLGIWGASGKLIATTAVPWNASWSNKSLEQIWNNMTWMEWDREINEYTSLIYSLIEESQNQQEKNEQELLELDKWASLWNWFNITNWLWY

SEQ ID NO: 80 - protein, 256 aa, synthetic construct
QLLSGIVQQQNNLLRAIEAQQHLLQLTVWGIKQLQARILGGSGGHTTWMEWDREIDNYTSLIYSLIEESQNQQEKNEQELLEGSSGGQLLSGIVQQQNNLLRAIEAQQHLLQLTVWGIKQLQARILGGSGGHTTWMEWDREIDNYTSLIYSLIEESQNQQEKNEQELLEGSSGGQLLSGIVQQQNNLLRAIEAQQHLLQLTVWGIKQLQARILGGSGGHTTWMEWDREINNYTSLIHSLIEESQNQQEKNEQELLE

SEQ ID NO: 81 - protein, 256 aa, synthetic construct
QLLSGIVQQQNNLLRAIEAQQHLLQLTVWGIKQLQARILGGSGGHTTWMEWDREIDNYTSLIYSLIEESQNQQEKNEQELLEGSSGGQLLSGIVQQQNNLLRAIEAQQHLLQLTVWGIKQLQARILGGSGGHTTWMEWDREIDNYTSLIYSLIEESQNQQEKNEQELLEGSSGGQLLSGIVQQQNNLLRAIEAQQHLLQLTVWGIKQLQARILGGSGGHTTWMEWDREIDNYTSLIYSLIEESQNQQEKNEQELLE

SEQ ID NO: 82 - protein, 269 aa, synthetic construct
MQLLSGIVQQQNNLLRAIEAQQHLLQLTVWGIKQLQARILGGSGGHTTWMEWDREINNYTSLIHSLIEESQNQQEKNEQELLEGSSGGQLLSGIVQQQNNLLRAIEAQQHLLQLTVWGIKQLQARILGGSGGHTTWMEWDREINNYTSLIHSLIEESQNQQEKNEQELLEGSSGGQLLSGIVQQQNNLLRAIEAQQHLLQLTVWGIKQLQARILGGRGGHTTWMEWDREINNYTSLIHSLIEESQNQQEKNEQELLGGLPETGHHHHHH

SEQ ID NO: 83 - protein, 275 aa, synthetic construct
MQLLSGIVQQQNNLLRAIEAQQHLLQLTVWGIKQLQARILGGSGGHTTWMEWDREIDNYTSLIYSLIEESQNQQEKNEQELLEGSSGGQLLSGIVQQQNNLLRAIEAQQHLLQLTVWGIKQLQARILGGSGGHTTWMEWDREIDNYTSLIYSLIEESQNQQEKNEQELLEGSSGGQLLSGIVQQQNNLLRAIEAQQHLLQLTVWGIKQLQARILGGSGGHTTWMEWDREINNYTSLIHSLIEESQNQQEKNEQELLEGGGLPETGSGGGHHHHHH

SEQ ID NO: 84 - protein, 274 aa, synthetic construct
MQLLSGIVQQQNNLLRAIEAQQHLLQLTVWGIKQLQARILGGSGGHTTWMEWDREIDNYTSLIYSLIEESQNQQEKNEQELLEGSSGGQLLSGIVQQQNNLLRAIEAQQHLLQLTVWGIKQLQARILGGSGGHTTWMEWDREIDNYTSLIYSLIEESQNQQEKNEQELLEGSSGGQLLSGIVQQQNNLLRAIEAQQHLLQLTVWGIKQLQARILGGSGGHTTWMEWDREIDNYTSLIYSLIEESQNQQEKNEQELLEGGGYRYRQGGGHHHHHH

SEQ ID NO: 85 - protein, 547 aa, synthetic construct; identical to SEQ ID NO: 18

SEQ ID NO: 86 - protein, 599 aa, synthetic construct; identical to SEQ ID NO: 19

SEQ ID NO: 87 - protein, 267 aa, synthetic construct
MQLLSGIVQQQNNLLRAIEAQQHLLQLTVWGIKQLQARILAGGSGGHTTWMEWDREINNYTSLIHSLIEESQNQQEKNEQELLEGSSGGQLLSGIVQQQNNLLRAIEAQQHLLQLTVWGIKQLQARILAGGSGGHTTWMEWDREINNYTSLIHSLIEESQNQQEKNEQELLEGSSGGQLLSGIVQQQNNLLRAIEAQQHLLQLTVWGIKQLQARILAGGRGGHTTWMEWDREINNYTSLIHSLIEESQNQQEKNEQELLGGHHHHHH

SEQ ID NO: 88 - protein, 146 aa, synthetic construct
TLTVQARQLLSGIVQQQNNELRAIEAQQHLLQLTVWGTKQLQARELAVERYLKDQQLLGIWGASGKLIATTAVPWNASWSNKSLEQIWNNMTWMEWDREINNYTSLIHSLIEESQNQQEKNEQELLELDKWASLWNWFNITNWLWY

SEQ ID NO: 89 - protein, 345 aa, synthetic construct
AVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNNMTWMEWDREINNYTSLIHSLIEESQNQQEKNEQELLELDKWASLWNWFNITNWLWYIKLFIMIVGGLVGLRIVFAVLSVVNRVRQGYSPLSFQTHLPIPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGAYRAIRHIPRRIRQGLERILL

SEQ ID NO: 90 - protein, 596 aa, synthetic construct
MKVAKDLVVSLAYQVRTEDGVLVDESPVSAPLDYLHGHGSLISGLETALEGHEVGDKFDVAVGANDAYGQYDENLVQRVPKDVFMGVDELQVGMRFLAETDQGPVPVEITAVEDDHVVVDGNHMLAGQNLKFNVEVVAIREATEEELAHGHVHGAHDHHHDHDHDGGGSGGGSGGGSGGGYRYRQGGGSGGGSGGGSGGGYRYRQGGGSGGGSGGGSGGGKVAKDLVVSLAYQVRTEDGVLVDESPVSAPLDYLHGHGSLISGLETALEGHEVGDKFDVAVGANDAYGQYDENLVQRVPKDVFMGVDELQVGMRFLAETDQGPVPVEITAVEDDHVVVDGNHMLAGQNLKFNVEVVAIREATEEELAHGHVHGAHDHHHDHDHDGGGSGGGSGGGSGGGYRYRQGGGSGGGSGGGSGGGYRYRQGGGSGGGSGGGSGGGSPKRLSAAIGKAWEEGVCGIRSATRLENIMWKQISNELNHILLENDMKFTVVVGDVAGILAQGKKMIRPQPMEHKYSWKSWGKAKIIGADVQNTTFIIDGPNTPECPDDQRAWNGGGSGGGSGGGSGGGLPETGGGGSGGGSGGGSGGGLEHHHHHH

SEQ ID NO: 91 - protein, 633 aa, synthetic construct
MKVAKDLVVSLAYQVRTEDGVLVDESPVSAPLDYLHGHGSLISGLETALEGHEVGDKFDVAVGANDAYGQYDENLVQRVPKDVFMGVDELQVGMRFLAETDQGPVPVEITAVEDDHVVVDGNHMLAGQNLKFNVEVVAIREATEEELAHGHVHGAHDHHHDHDHDGGGSGGGSGGGSGGGYRYRQGGGSGGGSGGGSGGGYRYRQGGGSGGGSGGGSGGGKVAKDLVVSLAYQVRTEDGVLVDESPVSAPLDYLHGHGSLISGLETALEGHEVGDKFDVAVGANDAYGQYDENLVQRVPKDVFMGVDELQVGMRFLAETDQGPVPVEITAVEDDHVVVDGNHMLAGQNLKFNVEVVAIREATEEELAHGHVHGAHDHHHDHDHDGGGSGGGSGGGSGGGYRYRQGGGSGGGSGGGSGGGYRYRQGGGSGGGSGGGSGGGEQYKFQADSPKRLSAAIGKAWEEGVCGIRSATRLENIMWKQISNELNHILLENDMKFTVVVGDVAGILAQGKKMIRPQPMEHKYSWKSWGKAKIIGADVQNTTFIIDGPNTPECPDDQRAWNIWEVEDYGFGIFTTNIWLKLRDSYTQVADGGGSGGGSGGGSGGGLPETGGGGSGGGSGGGSGGGLEHHHHHH

SEQ ID NO: 92 - protein, 609 aa, synthetic construct
MKVAKDLVVSLAYQVRTEDGVLVDESPVSAPLDYLHGHGSLISGLETALEGHEVGDKFDVAVGANDAYGQYDENLVQRVPKDVFMGVDELQVGMRFLAETDQGPVPVEITAVEDDHVVVDGNHMLAGQNLKFNVEVVAIREATEEELAHGHVHGAHDHHHDHDHDGGGSGGGSGGGYRYRQGGGSGGGSGGGYRYRQGGGSGGGSGGGKVAKDLVVSLAYQVRTEDGVLVDESPVSAPLDYLHGHGSLISGLETALEGHEVGDKFDVAVGANDAYGQYDENLVQRVPKDVFMGVDELQVGMRFLAETDQGPVPVEITAVEDDHVVVDGNHMLAGQNLKFNVEVVAIREATEEELAHGHVHGAHDHHHDHDHDGGGSGGGSGGGYRYRQGGGSGGGSGGGYRYRQGGGSGGGSGGGEQYKFQADSPKRLSAAIGKAWEEGVCGIRSATRLENIMWKQISNELNHILLENDMKFTVVVGDVAGILAQGKKMIRPQPMEHKYSWKSWGKAKIIGADVQNTTFIIDGPNTPECPDDQRAWNIWEVEDYGFGIFTTNIWLKLRDSYTQVADGGGSGGGSGGGSGGGLPETGGGGSGGGSGGGSGGGLEHHHHHH

SEQ ID NO: 93 - protein, 633 aa, synthetic construct
MKVAKDLVVSLAYQVRTEDGVLVDESPVSAPLDYLHGHGSLISGLETALEGHEVGDKFDVAVGANDAYGQYDENLVQRVPKDVFMGVDELQVGMRFLAETDQGPVPVEITAVEDDHVVVDGNHMLAGQNLKFNVEVVAIREATEEELAHGHVHGAHDHHHDHDHDEAAAKEAAAKEAAAKYRYRQEAAAKEAAAKEAAAKYRYRQEAAAKEAAAKEAAAKKVAKDLVVSLAYQVRTEDGVLVDESPVSAPLDYLHGHGSLISGLETALEGHEVGDKFDVAVGANDAYGQYDENLVQRVPKDVFMGVDELQVGMRFLAETDQGPVPVEITAVEDDHVVVDGNHMLAGQNLKFNVEVVAIREATEEELAHGHVHGAHDHHHDHDHDEAAAKEAAAKEAAAKYRYRQEAAAKEAAAKEAAAKYRYRQEAAAKEAAAKEAAAKEQYKFQADSPKRLSAAIGKAWEEGVCGIRSATRLENIMWKQISNELNHILLENDMKFTVVVGDVAGILAQGKKMIRPQPMEHKYSWKSWGKAKIIGADVQNTTFIIDGPNTPECPDDQRAWNIWEVEDYGFGIFTTNIWLKLRDSYTQVADGGGSGGGSGGGSGGGLPETGGGGSGGGSGGGSGGGLEHHHHHH

SEQ ID NO: 94 - protein, 633 aa, synthetic construct
MKVAKDLVVSLAYQVRTEDGVLVDESPVSAPLDYLHGHGSLISGLETALEGHEVGDKFDVAVGANDAYGQYDENLVQRVPKDVFMGVDELQVGMRFLAETDQGPVPVEITAVEDDHVVVDGNHMLAGQNLKFNVEVVAIREATEEELAHGHVHGAHDHHHDHDHDGGGSGGGSGGGSGGGRVRQRGGGSGGGSGGGSGGGRVRQRGGGSGGGSGGGSGGGKVAKDLVVSLAYQVRTEDGVLVDESPVSAPLDYLHGHGSLISGLETALEGHEVGDKFDVAVGANDAYGQYDENLVQRVPKDVFMGVDELQVGMRFLAETDQGPVPVEITAVEDDHVVVDGNHMLAGQNLKFNVEVVAIREATEEELAHGHVHGAHDHHHDHDHDGGGSGGGSGGGSGGGRVRQRGGGSGGGSGGGSGGGRVRQRGGGSGGGSGGGSGGGEQYKFQADSPKRLSAAIGKAWEEGVCGIRSATRLENIMWKQISNELNHILLENDMKFTVVVGDVAGILAQGKKMIRPQPMEHKYSWKSWGKAKIIGADVQNTTFIIDGPNTPECPDDQRAWNIWEVEDYGFGIFTTNIWLKLRDSYTQVADGGGSGGGSGGGSGGGLPETGGGGSGGGSGGGSGGGLEHHHHHH

SEQ ID NO: 95 - protein, 7 aa, synthetic construct
GRYESKG

SEQ ID NO: 96 - protein, 580 aa, synthetic construct
MKVAKDLVVSLAYQVRTEDGVLVDESPVSAPLDYLHGHGSLISGLETALEGHEVGDKFDVAVGANDAYGQYDENLVQRVPKDVFMGVDELQVGMRFLAETDQGPVPVEITAVEDDHVVVDGNHMLAGQNLKFNVEVVAIREATEEELAHGHVHGAHDHHHDHDHDGGGSGGGSGGGSGGGYRYRQGGGSGGGSGGGSGGGYRYRQGGGSGGGSGGGSGGGKVAKDLVVSLAYQVRTEDGVLVDESPVSAPLDYLHGHGSLISGLETALEGHEVGDKFDVAVGANDAYGQYDENLVQRVPKDVFMGVDELQVGMRFLAETDQGPVPVEITAVEDDHVVVDGNHMLAGQNLKFNVEVVAIREATEEELAHGHVHGAHDHHHDHDHDGGGSGGGSGGGSGGGYRYRQGGGSGGGSGGGSGGGYRYRQGGGSGGGSGGGSGGGSPKRLSAAIGKAWEEGVCGIRSATRLENIMWKQISNELNHILLENDMKFTVVVGDVAGILAQGKKMIRPQPMEHKYSWKSWGKAKIIGADVQNTTFIIDGPNTPECPDDQRAWNGGGSGGGLPETGGGGSGGGLEHHHHHH

SEQ ID NO: 97 - protein, 633 aa, synthetic construct
MKVAKDLVVSLAYQVRTEDGVLVDESPVSAPLDYLHGHGSLISGLETALEGHEVGDKFDVAVGANDAYGQYDENLVQRVPKDVFMGVDELQVGMRFLAETDQGPVPVEITAVEDDHVVVDGNHMLAGQNLKFNVEVVAIREATEEELAHGHVHGAHDHHHDHDHDEAAAKEAAAKEAAAKYRYRQGGGSGGGSGGGSGGGYRYRQEAAAKEAAAKEAAAKKVAKDLVVSLAYQVRTEDGVLVDESPVSAPLDYLHGHGSLISGLETALEGHEVGDKFDVAVGANDAYGQYDENLVQRVPKDVFMGVDELQVGMRFLAETDQGPVPVEITAVEDDHVVVDGNHMLAGQNLKFNVEVVAIREATEEELAHGHVHGAHDHHHDHDHDEAAAKEAAAKEAAAKYRYRQGGGSGGGSGGGSGGGYRYRQEAAAKEAAAKEAAAKEQYKFQADSPKRLSAAIGKAWEEGVCGIRSATRLENIMWKQISNELNHILLENDMKFTVVVGDVAGILAQGKKMIRPQPMEHKYSWKSWGKAKIIGADVQNTTFIIDGPNTPECPDDQRAWNIWEVEDYGFGIFTTNIWLKLRDSYTQVADGGGSGGGSGGGSGGGLPETGGGGSGGGSGGGSGGGLEHHHHHH

(19) United States
(12) Patent Application Publication
(10) Pub. No.: US 2024/0059746 A1
(43) Pub. Date: Feb. 22, 2024

(54) TRANSGLUTAMINASE SUBSTRATES FOR LABELING

(71) Applicant: Roche Diagnostics Operations, Inc., Indianapolis, IN (US)

(72) Inventors: Juliane Benz, Penzberg (DE); Simon Jochum, Penzberg (DE); Matthias Hemann, Penzberg (DE)

(21) Appl. No.: 18/506,022

(22) Filed: Nov. 9, 2023

Related U.S. Application Data
(63) Continuation-in-part of application No. PCT/EP2023/061443, filed on May 2, 2023.

(30) Foreign Application Priority Data
May 3, 2022 (EP) 22171401.7

(51) Int. Cl. C07K 14/435; C07K 14/005; G01N 33/569; G01N 33/58

(52) U.S. Cl. CPC C07K 14/435; C07K 14/005; G01N 33/56988; G01N 33/58; G01N 33/581; C07K 2319/40; G01N 2333/16; G01N 2469/20

(57) ABSTRACT

Fusion polypeptides are disclosed which are substrates for Kutzneria albida transglutaminase. The fusion polypeptides comprise one or more FKBP chaperone(s) and a target polypeptide. Each of these elements is separated from the neighboring element by a linker amino acid sequence. It was found that inserting glutamic acid containing transglutaminase recognition motifs into the linker amino acid chains is advantageous. Subsequent labeling reactions catalyzed by the transglutaminase surprisingly provide labeled fusion polypeptides have superior properties when compared with chemically random-labeled fusion polypeptides of similar design. Assays and kits are provided for in vitro detection of target antibodies in samples.

### BACKGROUND

The present disclosure is about immunoassays which detect in a sample antibodies against a particular antigen. Antibody isotypes found in blood, such as, but not limited to, IgG, IgE, IgD, and single IgA are bivalent, that is to say each have two antigen binding sites. Each binding site is capable of binding to the target antigen of the antibody. There is a general need to detect particular antigen-specific antibodies in samples. By way of example, an antibody specific for an antigen that is derived from a pathogen can indicate exposure to the pathogen. Detection in vitro of such an antibody in a sample obtained from a patient can provide particular medical value in the diagnosis of a disease which is caused by the pathogen.

An immunoassay to detect an antibody makes use of this feature. In a generic aspect, such an assay provides an antigen which is contacted with a sample suspected of containing an antibody specific for and capable of binding to the antigen. If the antibody is present in the sample, it immunoreacts with the antigen to form a complex, the immunoreaction product. In this complex one antigen binding site of the antibody attaches to the antigen by physical interaction. The immunoassay detects such immunoreaction products, i.e. complexes.

Detection can be done by using a label. The antigen can be labeled, and immunoreaction products containing labeled antigen are detected. In a specific embodiment, immunoreaction products containing labeled antigen are separated from unbound labeled antigen and sample material which has not taken part in the immunoreaction. If antigen has been present in the sample, label is separated with the immunoreaction products. In this case, detection of label indicates the presence of immunoreaction products and hence presence of the target antibody in the sample.

An important precondition must be met for the immunoreaction to take place: Antibody detection in vitro requires stabilization of the antigen which is presented to the antibody in the sample. A desired stabilized antigen maintains its conformation and solubility, thereby securing its capability of binding target antibody in a reproducible way, and specifically under conditions of an in vitro test setting.

To this end, technical challenges exist in several aspects, particularly considering antigens derived from peptides or polypeptides. In particular, production of purified preparations of antigens for immunoassays can be hampered by solubility issues as certain antigens tend to aggregate and eventually precipitate. In such a case the required amount of antigen for an immunoassay may not be met. Also, purified antigens in vitro may not sufficiently reflect the original antigen in vivo, due to alterations in their conformation. In such cases the ability to bind to a target antibody in vitro becomes compromised.

Further challenges exist with respect to the labeling of antigen which is a peptide or polypeptide chain (i.e. an amino acid sequence). Attachment of a label is frequently made using chemical reactions. A typical example is amine-reactive crosslinker chemistry by which the label and primary amine groups of the antigen are coupled, very often using N-hydroxysuccinimide esters. Primary amines exist at the N-terminus of each polypeptide chain and in the side-chain of lysine (Lys, K) amino acid residues. As a matter of its chemical principle, there is hardly any site specificity for such chemical reactions. Reacting amine groups of the antigen chemically and attaching label to these groups may mask a critical part of the antigen such that the binding site of the antibody cannot bind anymore. While an excess of crosslinking may in the extreme lead to complete functional inactivation of the antigen (i.e. no antibody binding is possible, anymore), titration experiments are required to determine suitable concentrations of label, crosslinking reagent and antigen. As a result, label density on an antigen is not always optimal, and following a crosslinking reaction there is always a distribution of antigen which is either unlabeled, or labeled in excess, or labeled at higher densities or labeled at lower densities. Depending on the severity of undesired labeling effects, the detection process in an immunoassay may become compromised and requires laborious and time-consuming optimization effort.

FK506 binding proteins (FKBPs) have been identified in many eukaryotes from yeast to humans and function as protein folding chaperones for proteins containing proline residues. An example for prokaryotic FKBP-type polypeptide is SlyD. The bacterial slyD gene (exemplarily from E. coli) encodes a FKBP-type peptidyl-prolyl cis-trans isomerase (PPIase). SlyD is a bacterial two-domain protein that functions as a molecular chaperone, a prolyl cis/trans isomerase, and a nickel-binding protein. The chaperone function located in one domain of SlyD is involved in twin-arginine translocation and increases the catalytic efficiency of the prolyl cis/trans isomerase domain in protein folding by two orders of magnitude. Non-limiting examples for such FKBP-type chaperones are presented herein as SEQ ID NO: 1, SEQ ID NO: 2, SEQ ID NO: 3 and SEQ ID NO: 4. Further examples are documented in the literature, e.g. by Zoldák G. & Schmid F. X. (2011) J Mol Biol 406, 176-194 and Scholz C. et al. (2006) Biochemistry 45, 20-33, and elsewhere.

Issues regarding antigen solubility and conformation have been addressed previously. It has been reported in WO2003000877A2 that folding helpers, e.g., many members of the peptidyl prolyl isomerase (PPI) class, especially from the family of FKBP-type proteins, not only exhibit catalytic activity, but also bring about desired effects on solubility of polypeptides which otherwise tend to aggregate. They do so by forming soluble complexes with such polypeptides that in an unchaperoned and isolated form are prone to aggregation. Such polypeptides that are otherwise hardly soluble or insoluble under physiological conditions turn out to be soluble under mild physiological conditions (i.e. without need for solubilizing additives such as detergents or chaotropic agents) once they are bound in a complex with the appropriate PPI chaperone.

WO2007077008A1 reports a chimaeric fusion polypeptide comprising a polypeptide sequence containing the polypeptide binding segment of a non-human chaperone protein, a polypeptide sequence of an FKBP polypeptide or a FKBP-like domain that is fused to the N-terminal end of the non-human chaperone polypeptide sequence, and a polypeptide sequence of a FKBP polypeptide or a FKBP-like domain that is fused to the C-terminal end of the non-human chaperone polypeptide sequence.

EP1780282A1 discloses the finding that instead of by forming a complex with an FKBP chaperone, enhanced solubilization and reduced aggregation of the antigen without solubilizing additives can advantageously be achieved by joining the antigen and the FKBP chaperone in a fusion polypeptide. The document reports a fusion polypeptide comprising the amino acid sequence of a specific Rubella E1 antigen which is N-terminally fused to a FKBP chaperone amino acid sequence. EP2127679A1 reports a recombinantly produced fusion polypeptide comprising at least one amino acid sequence corresponding to a SlpA chaperone, and at least one amino acid sequence corresponding to a target polypeptide.

As it becomes clear, technical problems concerning undesired aggregation and precipitation of purified antigen can be mitigated by providing the antigen recombinantly as a target polypeptide amino acid sequence in a fusion polypeptide wherein to the N-terminal portion of the target there is or are appended one or several copies of a FKBP chaperone amino acid sequence. In practice, the target polypeptide and each of the FKBP chaperone domains are joined by coupling amino acid sequences which function as linkers between any of these elements and the adjacent element in the fusion polypeptide. However, in view of attaching label to such fusion polypeptides by way of chemical reactions, technical challenges persist. Also in the case of a fusion polypeptide chemical crosslinking can mask critical part of the antigen. In addition, chemical crosslinking also affects the FKBP chaperone portion. As a consequence, the desired function of the chaperone can be compromised, and hence the stability of the antigen portion of the fusion polypeptide. In addition the problem of randomized distribution of label persists. Conventional chemical strategies for polypeptide modification are difficult to control and give rise to heterogeneous populations of labeled polypeptides with variable stoichiometries, with each member in a population having its own in vitro characteristics. Furthermore, the number of chemically reacted target polypeptides or label can not be controlled, and thus is not defined, and usually follows a Poisson distribution, which causes problems when desiring to quantify reactions.

For at least these reasons and others there is demand in the art to provide better alternatives of providing stabilized target polypeptides such as antigens for immunoassays.

The discovery of a novel transglutaminase (Kutzneria albida transglutaminase; KalbTGase) and the identification of the respective peptide substrates have been described (Steffen et al. (2017) J Biol Chem 292, 15622-15635). KalbTGase catalyzes the formation of an isopeptide bond between an donor acyl group, particularly a of a glutamine (Gln, Q) side chain and an alkyl amine donor group, e.g. of a lysine (Lys, K) side chain. The motifs YRYRQ (SEQ ID NO: 20) and RVRQR (SEQ ID NO: 21) are particularly suited KalbTGase glutamine-containing-motifs (Qtag, Qtags). Steffen et al. (2017), supra, teport that RYESK (SEQ ID NO: 73) as a KalbTGase lysine-containing-acceptor-motif (Ktag). However, KalbTGase is capable of processing a variety of other amine donor groups as Ktags, in addition to the lysine side chain. An example therefor is biotin-dPEG(23)-NH₂ (Steffen et al. (2017), supra).

KalbTGase-mediated introduction of artificial, bio-orthogonal groups for site-specific and stoichiometric polypeptide modification potentially offers a solution to problems discussed above. However, the necessarily required insertion into a target polypeptide such as an antigen amino acid sequence is expected to change its conformation, unless laborious effort is invested to determine permissive sites for insertion of a Qtag amino acid sequence. By the same token, insertion of a Qtag motif into an amino acid sequence of an FKBP chaperone raises similar concerns.

### BRIEF SUMMARY

The inventors found for fusion polypeptides that inserting a Qtag into a coupling amino acid sequence which is located between two adjacent FKBP chaperones is well suited for providing an attachment site for KalbTGase-mediated coupling of Ktags. In addition, the inventors found that inserting a Qtag into a coupling amino acid sequence which is located between a FKBP chaperones and an adjacent target polypeptide is also well suited for providing an attachment site for KalbTGase-mediated coupling of Ktags. Such Qtag insertions not only provide for bio-orthogonal addition of label in a site-specific and stoichiometric manner. It could be shown that surprisingly specific fusion polypeptides which are KalbTGase substrates and which are labeled using this enzyme markedly increase signal-to-noise ratio in label detection. In addition, such labeled substrate show a surprising increase in temperature stability.

In a first aspect the present report provides a recombinant KalbTGase substrate comprising a fusion polypeptide of Formula I,

_{N-terminus}[Aⁿ-Lⁿ-]ₙB_{C-terminus} (Formula I),
wherein
a hyphen “-” denotes a peptide bond;
n is an integer number from 1 to 6;
a pair of square brackets “[” and “]” delimits N-terminal and C-terminal borders of an amino acid sequence which is present n times;
Aⁿ is a FKBP chaperone amino acid sequence, and in case of n>1, each Aⁿ is an independently selected FKBP chaperone amino acid sequence;
Lⁿ is a coupling amino acid sequence, and in case of n>1, each Lⁿ is an independently selected coupling amino acid sequence,
B is a target polypeptide amino acid sequence;
and wherein
each Lⁿ is of Formula IIa or Formula IIb

T[-Qtag^{m}-R^{m}]ₘ (Formula IIa),

[R^{m}-Qtag^{m}-]ₘT (Formula IIb);
wherein
m is an integer number from 1 to 10;
a pair of square brackets “[” and “]” delimits N-terminal and C-terminal borders of an amino acid sequence which is present m times;
T is a flexible or rigid linker amino acid sequence comprising 5 to 500 amino acids;
R^{m} is a flexible or rigid linker amino acid sequences comprising 5 to 500 amino acids, and in case of m>1, each R^{m} is an independently selected flexible or rigid linker amino acid sequences comprising 5 to 500 amino acids;
Qtag^{m} is an amino acid sequence motif containing an acyl donor glutamine residue for KalbTGase transglutaminase activity, and in case of m>1, each Qtag^{m} is an independently selected amino acid sequence motif containing an acyl donor glutamine residue for KalbTGase transglutaminase activity;
with the proviso that SEQ ID NO: 18 and SEQ ID NO: 19 are excluded from Formula I.

In a second aspect the present report provides a method of forming a target polypeptide with a covalently attached label, the method comprising the steps of
a. providing a KalbTGase substrate according to the first aspect and all its embodiments herein, the KalbTGase substrate comprising the target polypeptide and one or more acyl donor glutamine residue(s) for KalbTGase transglutaminase activity;
b. providing a label conjugate, wherein in the label conjugate the label is covalently attached to a Ktag, wherein the Ktag is a KalbTG lysine-containing-acceptor-motif or a functional analog thereof, wherein the Ktag comprises a primary amine group capable of being reacted with an acyl donor glutamine residue for KalbTGase transglutaminase activity in the presence of KalbTGase;
c. contacting the fusion polypeptide of (a) and the conjugate of the label of (b) with KalbTGase under conditions allowing transglutaminase activity to occur; thereby reacting an acyl donor glutamine residue with a primary amine group to form a covalent connection,
   thereby forming the target polypeptide with the covalently attached label.

In a third aspect the present report provides a method of forming a target polypeptide with a covalently attached capture group, the method comprising the steps of
a. providing a KalbTGase substrate according to the first aspect and all its embodiments herein, the KalbTGase substrate comprising the target polypeptide and one or more acyl donor glutamine residue(s) for KalbTGase transglutaminase activity;
b. providing a capture group conjugate, wherein in the capture group conjugate the capture group is covalently attached to a Ktag, wherein the Ktag is a KalbTG lysine-containing-acceptor-motif or a functional analog thereof, wherein the Ktag comprises a primary amine group capable of being reacted with an acyl donor glutamine residue for KalbTGase transglutaminase activity in the presence of KalbTGase;
c. contacting the fusion polypeptide of (a) and the conjugate of the capture group of (b) with KalbTGase under conditions allowing transglutaminase activity to occur;
   thereby reacting an acyl donor glutamine residue with a primary amine group to form a covalent connection,
   thereby forming the target polypeptide with the covalently attached capture group.

In a fourth aspect the present report provides a labeled target polypeptide, obtained or obtainable by the method according to the second aspect and all its embodiments herein.

In a fifth aspect the present report provides a target polypeptide with a covalently attached capture group, obtained or obtainable by the method according to the third aspect and all its embodiments herein.

In a sixth aspect the present report provides a composition suitable for detecting in an isolated sample target antibodies specific for an antigen amino acid sequence, wherein the composition comprises a labeled target polypeptide according to the fourth aspect and all its embodiments herein, wherein the antigen amino acid sequence is comprised in the labeled target polypeptide.

In a seventh aspect the present report provides a method for detecting in an isolated sample a target antibody (═X) specific for an antigen amino acid sequence (═Y), said method comprising
a. forming an immunoreaction mixture by admixing a body fluid sample suspected of containing X with a labeled target polypeptide according to the fourth aspect and all its embodiments herein, wherein Y is comprised in the labeled target polypeptide,
b. maintaining the immunoreaction admixture of step (a) for a time period sufficient for allowing X present in the body fluid sample to immunoreact with Y comprised in the labeled target polypeptide to form an immunoreaction product (═X:Y) in the admixture; and
c. detecting the presence and/or the concentration of X:Y formed in step (b);
   thereby detecting in the isolated sample target antibody specific for the antigen amino acid sequence.

In an eighth aspect the present report provides the use of (i) a KalbTGase substrate according to the first aspect and all its embodiments herein, (ii) a label conjugate and (iii) KalbTGase for producing a labeled target polypeptide, wherein in the label conjugate the label is covalently attached to a Ktag, wherein the Ktag is a KalbTG lysine-containing-acceptor-motif or a functional analog thereof, wherein the Ktag comprises a primary amine group capable of being reacted with an acyl donor glutamine residue for KalbTGase transglutaminase activity in the presence of KalbTGase.

In a ninth aspect the present report provides the use of a labeled target polypeptide obtained or obtainable by the method according to the fourth aspect and all its embodiments herein for detecting in an isolated sample target antibodies specific for an antigen amino acid sequence.

In a tenth aspect the present report provides the use of a labeled target polypeptide according to the fourth aspect and all its embodiments herein and a separate target polypeptide with a covalently attached capture group according to the fifth aspect and all its embodiments herein for detecting in an isolated sample target antibodies specific for an antigen amino acid sequence.

In an eleventh aspect the present report provides a kit of parts for detecting in an isolated sample target antibodies specific for an antigen amino acid sequence, the kit containing a labeled target polypeptide according to the fourth aspect and all its embodiments herein, the labeled target polypeptide comprising the antigen amino acid sequence.

In a twelfth aspect the present report provides a DNA encoding a fusion polypeptide comprising a target polypeptide and one or more acyl donor glutamine residue(s) for KalbTGase transglutaminase activity, wherein a codon for N-terminal methionine is appended to a nucleotide sequence encoding the amino acid sequence of the KalbTGase substrate according to the first aspect and all its embodiments herein.

In a thirteenth aspect the present report provides an expression vector for recombinant expression in a transformed organism, the expression vector comprising the DNA according to the twelfth aspect and all its embodiments herein.

In a fourteenth aspect the present report provides a prokaryotic host organism stably transformed with an expression vector according to the thirteenth aspect and all its embodiments herein, wherein the transformed host organism is capable of expressing the encoded KalbTGase substrate from the expression vector

In a fifteenth aspect the present report provides a method of producing a recombinant KalbTGase substrate, the method comprising the steps of
(a) providing and culturing the transformed host organism according to the fourteenth aspect and all its embodiments herein,
(b) expressing in the transformed host organism of (a) the recombinant KalbTGase substrate, and
(c) purifying the expressed recombinant KalbTGase substrate from the transformed host organism, thereby producing the recombinant KalbTGase substrate.

### DESCRIPTION OF THE FIGURES

FIG. 1 depicts a Ktag with a Ru label.

FIG. 2 depicts a Ktag with a biotin group.

FIG. 3 depicts a chromatogram of a size exclusion chromatography which is performed to separate the products after a Ru-labeling reaction in the presence of KalbTGase.

FIG. 4 depicts in A a Superdex® 200 chromatogram of a purified fusion polypeptide with a biotin capture group, attached using KalbTGase; depicts in B a Superdex® 200 chromatogram of a purified fusion polypeptide with Ruthenium label, attached using KalbTGase.

### DETAILED DESCRIPTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The various described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

The terms “a”, “an” and “the” generally include plural referents, unless the context clearly indicates otherwise. As used herein, “plurality” is understood to mean more than one. For example, a plurality refers to at least two, three, four, five, or more. Unless specifically stated or obvious from context, as used herein, the term “or” is understood to be inclusive.

“Directly repeated” refers to repetition without further interruption or any further intervening amino acid sequence.

“Functional variant” refers to a polypeptide with a similar function as the non-varied reference polypeptide. In an embodiment a functional variant of a FKBP chaperone amino acid sequence preserves chaperone function. A “Functional variant” also encompasses appended peptide sequences such as a histidine-tag or a N- or C-terminal sequence alteration which might be the case as a cloning artifact.

“N-terminal and C-terminal” refer to the respective end of an element present in a fusion polypeptide, or the fusion polypeptide as a whole.

The term “target polypeptide” refers to any polypeptide of interest, provided that the polypeptide comprises one or more antigenic determinants.

“Linker amino acid sequence” refers to flexible and rigid linker amino acid sequence as disclosed and discussed by Chen X. Et al in (2013) Adv Drug Deliv Rev. 65, 1357-1369.

The term “recombinant” refers to an amino acid sequence or a nucleotide sequence that has been intentionally modified by recombinant methods. By the term “recombinant nucleic acid” herein is meant a nucleic acid, originally formed in vitro, in general, by the manipulation of a nucleic acid by endonucleases, in a form not normally found in nature. Thus an isolated, mutant DNA polymerase nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell, it will replicate non-recombinantly, i.e., using the in vivo cellular machinery of the host cell rather than in vitro manipulations; however, such nucleic acids, once produced recombinantly, although subsequently replicated non-recombinantly, are still considered recombinant for the purposes of the invention. A “recombinant polypeptide” or “recombinantly produced polypeptide” is a polypeptide made using recombinant techniques, i.e., through the expression of a recombinant nucleic acid as explained above.

The term “vector” refers to a piece of DNA, typically double-stranded, which may have inserted into it a piece of foreign DNA. The vector or may be, for example, of plasmid origin. Vectors contain “replicon” polynucleotide sequences that facilitate the autonomous replication of the vector in a host cell. Foreign DNA is defined as heterologous DNA, which is DNA not naturally found in the host cell, which, for example, replicates the vector molecule, encodes a selectable or screenable marker, or encodes a transgene. The vector is used to transport the foreign or heterologous DNA into a suitable host cell. Once in the host cell, the vector can replicate independently of or coincidental with the host chromosomal DNA, and several copies of the vector and its inserted DNA can be generated. In addition, the vector can also contain the necessary elements that permit transcription of the inserted DNA into an mRNA molecule or otherwise cause replication of the inserted DNA into multiple copies of RNA. Some expression vectors additionally contain sequence elements adjacent to the inserted DNA that increase the half-life of the expressed mRNA and/or allow translation of the mRNA into a protein molecule. Many molecules of mRNA and polypeptide encoded by the inserted DNA can thus be rapidly synthesized.

The term “fusion polypeptide” refers to a polypeptide consisting of a linear sequence of two or more building blocks, wherein each building block of the fusion polypeptide is a peptide or a polypeptide, and wherein two adjacent building blocks are connected by a peptide bond. A fusion polypeptide can be produced recombinantly as a contiguous translation product in vivo or in vitro. Alternatively, a fusion polypeptide can be provided using chemical synthesis ex vivo. Its use as a folding helper for target polypeptides is disclosed, specifically as an additive to an immunoassay mixture. “Target polypeptide” refers to any polypeptide of interest, provided that the polypeptide comprises one or more antigenic determinants.

The term “integer number” refers to a positive natural number which in a specific embodiment is selected from 0, 1, 2, 3, 4, 5, 6, 7, 8, 9, 10, and any subset thereof.

The term “sample”, refers to a part or piece of a tissue, organ or individual, typically being smaller than such tissue, organ or individual, intended to represent the whole of the tissue, organ or individual. Upon analysis a sample provides information about the tissue status or the health or diseased status of an organ or individual. Examples of samples include but are not limited to fluid samples such as blood, serum, plasma, synovial fluid, urine, saliva, and lymphatic fluid, or solid samples such as tissue extracts, cartilage, bone, synovium, and connective tissue. Analysis of a sample may be accomplished on a visual or chemical basis. Visual analysis includes but is not limited to microscopic imaging or radiographic scanning of a tissue, organ or individual allowing for morphological evaluation of a sample. Chemical analysis includes but is not limited to the detection of the presence or absence of specific indicators or alterations in their amount, concentration or level. The sample is an in vitro sample, isolated from a body, it will be analyzed in vitro and not transferred back into the body.

The term “measurement”, “measuring”, “detecting” or “detection” preferably comprises a qualitative, a semi-quantitative or a quantitative measurement. The term “detecting the presence” refers to a qualitative measurement, indicating the presence of absence without any statement to the quantities (e.g. yes or no statement). The term “detecting amount” refers to a quantitative measurement wherein the absolute number is detected (ng). The term “detecting the concentration” refers to a quantitative measurement wherein the amount is determined in relation to a given volume (e.g. ng/ml).

The term “antigen” is a molecule or molecular structure, which is bound to by an antigen-specific antibody (Ab) or B cell antigen receptor (BCR). The presence of an antigen in the body normally triggers an immune response. In the body, each antibody is specifically produced to match an antigen after cells of the immune system come into contact with it; this allows a precise identification or matching of the antigen and the initiation of a tailored response. In most cases, an antibody can only react to and bind one specific antigen; in some instances, however, antibodies may cross-react and bind more than one antigen. Antigens are normally proteins, peptides (linker amino acid sequences) and polysaccharides (chains of mono-saccharides/simple sugars) or combinations thereof. For the present invention, an antigen is used as a specific ingredient in an immunoassay that specifically binds to antibodies that are present in the analyzed sample and that bind to the antigen.

The term “chaperone” refers to protein folding helpers which assist the folding and maintenance of the structural integrity of other proteins. Along with cyclophilin, FKBPs belong to the immunophilin family. In the human genome there are encoded fifteen proteins whose segments have significant homology with the sequence of 12 kDa protein which is the target of the potent immunosuppressive macrolides FK506 or rapamycin. The 12 kDa archetype of the FK506-binding protein (FKBP), known as FKBP-12, is an abundant intracellular protein. FKBP12 functions as a PPIase that catalyzes interconversion between prolyl cis/trans conformations. FKBPs are involved in diverse cellular functions including protein folding, cellular signaling, apoptosis and transcription. They elicit their function through direct binding and altering conformation of their target proteins, hence acting as molecular switches. Examples of folding helpers are described in detail in WO2003000877. Exemplified, chaperones of the peptidyl prolyl isomerase class such as chaperones of the FKBP family can be used for fusion to the antigen variants. Examples of FKBP chaperones suitable as fusion partners are FkpA (aa 26-270, UniProt ID P45523), SlyD (1-165, UniProt ID P0A9K9) and SlpA (2-149, UniProt ID P0AEM0). A further chaperone suitable as a fusion partner is Skp (21-161, UniProt ID P0AEU7), a trimeric chaperone from the periplasm of E. coli, not belonging to the FKBP family. It is not always necessary to use the complete sequence of a chaperone. Functional fragments of chaperones (so-called binding-competent modules) which still possess the required abilities and functions may also be used (cf. WO199813496). “FKBP chaperone” particularly refers to a FKBP-type peptidyl prolyl isomerase. Specific advantageous use thereof as part of a fusion polypeptide is reported in WO2003000878, EP1780282, WO2007077008, EP2127679, WO2012150320, WO2014072305, and WO2014072306. Specific but non-limiting examples of FKBP chaperones are FkpA, SlyD, SlyD-like protein, trigger factor, and functionally active variants thereof, including truncations, amino acid deletions, insertions or replacements, and affinity-tagged (e.g. with a His-tag) versions thereof.

The term “peptide” refers to a chain of at least two and less than ten amino acids that are linked together by peptide bonds.

“Amino acid” refers to any monomer unit that can be incorporated into a peptide, polypeptide, or protein. As used herein, the term “amino acid” includes the following twenty natural or genetically encoded alpha-amino acids: alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V).

“Peptide bond” refers to an amide-type bond between the α-carboxyl group of a first amino acid and the α-amino group of a second amino acid. The “peptide bond” is different from the covalent bond generated by transglutaminase activity in that the latter is an isopeptide bond.

The term “coupling amino acid sequence” refers to a stretch of amino acids located between two FKBP chaperone amino acid sequences or between a FKBP chaperone amino acid sequence and a target polypeptide. In the present disclosure a coupling amino acid sequence contains one or more Qtags. “Element” refers to a building block or a group of two or more building blocks being part of a fusion polypeptide, including a connecting bond between adjacent building blocks. Building blocks can be identical or different. For the purpose of the present disclosure, elements include “T”, “R^{m}” “Lⁿ”, “Aⁿ”, “B”, “Qtag^{m}”.

“KalbTGase” refers to Kutzneria albida transglutaminase as described by Steffen W. et al. (2017) J Biol Chem 292, 15622-15635. In an embodiment KalbTGase is the polypeptide of SEQ ID NO: 65.

“KalbTGase substrate” refers to a polypeptide capable of undergoing a transglutaminase reaction catalyzed by KalbTGase, wherein an isopeptide bond between an donor acyl group, particularly a of a glutamine (Gln, Q) side chain, and an alkyl amine donor group, is formed. This effect can be seen clearly in immunoassays aimed at detecting antibodies which bind to an antigen comprised in a fusion polypeptide, wherein the fusion polypeptide contains one or more Qtag(s) having label covalently attached thereto by means of a Ktag and KalbTGase catalytic activity.

The inventors found for fusion polypeptides that inserting a Qtag into a coupling amino acid sequence which is located between two adjacent FKBP chaperones is well suited for providing an attachment site for KalbTGase-mediated coupling of Ktags. In addition, the inventors found that inserting a Qtag into a coupling amino acid sequence which is located between a FKBP chaperones and an adjacent target polypeptide is also well suited for providing an attachment site for KalbTGase-mediated coupling of Ktags. Thus, in a first aspect the present report provides a recombinant KalbTGase substrate comprising a fusion polypeptide of Formula I,

_{N-terminus}[Aⁿ-Lⁿ-]ₙB_{C-terminus} (Formula I),
wherein
a hyphen “-” denotes a peptide bond;
n is an integer number from 1 to 6;
a pair of square brackets “[” and “]” delimits N-terminal and C-terminal borders of an amino acid sequence which is present n times;
Aⁿ is a FKBP chaperone amino acid sequence, and in case of n>1, each Aⁿ is an independently selected FKBP chaperone amino acid sequence;
Lⁿ is a coupling amino acid sequence, and in case of n>1, each Lⁿ is an independently selected coupling amino acid sequence,
B is a target polypeptide amino acid sequence;
and wherein
each Lⁿ is of Formula IIa or Formula IIb

T[-Qtag^{m}-R^{m}]ₘ (Formula IIa),

[R^{m}-Qtag^{m}-]ₘT (Formula IIb);
wherein
m is an integer number from 1 to 10;
a pair of square brackets “[” and “]” delimits N-terminal and C-terminal borders of an amino acid sequence which is present m times;
T is a flexible or rigid linker amino acid sequence comprising 5 to 500 amino acids;
R^{m} is a flexible or rigid linker amino acid sequences comprising 5 to 500 amino acids, and in case of m>1, each R^{m} is an independently selected flexible or rigid linker amino acid sequences comprising 5 to 500 amino acids; and
Qtag^{m} is an amino acid sequence motif containing an acyl donor glutamine residue for KalbTGase transglutaminase activity, and in case of m>1, each Qtag^{m} is an independently selected amino acid sequence motif containing an acyl donor glutamine residue for KalbTGase transglutaminase activity.

In a specific embodiment of this aspect SEQ ID NO: 18 and SEQ ID NO: 19 are excluded from Formula I.

In an embodiment of the recombinant KalbTGase substrate, the first aspect Aⁿ is selected from the group consisting of SEQ ID NO: 4, SEQ ID NO: 5, and SEQ ID NO: 6, or a functional variant thereof with a sequence identity of 85% or higher. In more specific embodiments of the recombinant KalbTGase substrate, the sequence identity is any of 90%, 95%, and 99%. In another embodiment of the recombinant KalbTGase substrate, all Aⁿ are derived from a single member of the group consisting of SEQ ID NO: 4, SEQ ID NO: 5, and SEQ ID NO: 6, and the Aⁿ amino acid sequences are at least 90% identical. In yet another embodiment of the recombinant KalbTGase substrate, each Aⁿ amino acid sequence functionally preserves FKBP chaperone activity in the fusion polypeptide. In yet another embodiment of the recombinant KalbTGase substrate, the integer number n is 2 to 4.

In an embodiment of the recombinant KalbTGase substrate, B is an antigen amino acid sequence. In a specific embodiment, of the recombinant KalbTGase substrate, the antigen amino acid sequence is derived from a peptide or polypeptide originating from a pathogen of the group consisting of a mammalian pathogenic virus, a mammalian pathogenic bacterium, a mammalian pathogenic single- or multi-cell parasite, a mammalian cancer cell cancer cell, and a prion. In a more specific embodiment of the recombinant KalbTGase substrate, the antigen amino acid sequence is derived from a peptide or polypeptide originating from a member of the group consisting of human immunodeficiency virus, vaccinia virus, rubella virus, polio virus, adenovirus, influenza virus, hepatitis A virus, hepatitis B virus, hepatitis C virus, dengue virus, Japanese B encephalitis virus, Varicella zoster virus, cytomegalovirus, herpes simplex virus, herpes genitalis virus, Epstein-Barr virus, rotavirus, chikungunya virus, west-nile virus, tick-borne encephalitis virus, zika virus, yellow fever virus, Marburg virus, Ebola virus, measles virus, mumps virus, rabies virus, MERS coronavirus, SARS coronavirus, and SARS coronavirus-2. In yet another more specific embodiment of the recombinant KalbTGase substrate, B comprises the amino acid sequence of SEQ ID NO: 76 or SEQ ID NO: 77. In yet another more specific embodiment of the recombinant KalbTGase substrate, the antigen amino acid sequence is derived from a peptide or polypeptide originating from a member of the group consisting of Vibrio cholerae, Salmonella typhimurium, Salmonella typhi, Shigella dysenteriae, Shigella flexneri, Shigella boydii, Shigella sonnei, Helicobacter pylori, Bordetella pertussis, Streptococcus pyogenes, Streptococcus pneumoniae, Haemophilus influenzae, Clostridium tetani, Corynebacterium diphtheriae, Mycobacterium tuberculosis, Mycobacterium leprae, Rickettsia rickettsii, Rickettsia conorii, Rickettsia japonica, Rickettsia akari, Treponema pallidum, Neisseria gonorrhoeae, Neisseria meningitidis, Coccidioides immitis, Toxoplasma gondii, Borrelia afzelii, Borrelia garinii, Borrelia burgdorferi, Entamoeba histolytica, Plasmodium falciparum, Plasmodium spec., and Trypanosoma cruzi.

In an embodiment of the recombinant KalbTGase substrate, Qtag^{m} is selected from the group consisting of SEQ ID NO: 20, SEQ ID NO: 21, SEQ ID NO: 22, SEQ ID NO: 23, SEQ ID NO: 22, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, and SEQ ID NO: 64. In an embodiment of the recombinant KalbTGase substrate, wherein m is 2 to 10, each Qtag^{m} is independently selected from the group consisting of SEQ ID NO: 20, SEQ ID NO: 21, SEQ ID NO: 22, SEQ ID NO: 23, SEQ ID NO: 22, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, and SEQ ID NO: 64. In a more specific embodiment of the recombinant KalbTGase substrate, Qtag^{m} is selected from YRYRQ (SEQ ID NO: 20) and RVRQR (SEQ ID NO: 21), or in case of m>1, each Qtag^{m} is independently selected from the group consisting of YRYRQ (SEQ ID NO: 20) and RVRQR (SEQ ID NO: 21). In yet an even more specific embodiment of the recombinant KalbTGase substrate, wherein all Qtag^{m} are identical.

In an embodiment of the recombinant KalbTGase substrate, R^{m} and T are selected from (i) a flexible linker amino acid sequence composed of glycine (G) and optionally
serine (S), and
(ii) a rigid linker amino acid sequence composed of glutamic acid (E), alanine (A), and lysine (K).

In an embodiment of the recombinant KalbTGase substrate, at least one flexible linker amino acid sequence of T and/or R^{m} is an independently selected amino acid sequence of Formula III

[Gₜ-[S]ᵣ]ₚ-[G]_{q} (Formula III),
wherein
a pair of square brackets “[” and “]” delimits N-terminal and C-terminal borders of an amino acid sequence which is present p or q times;
t is an integer from 1 to 5, indicating the number of consecutive glycines in case t is any of 2 to 5;
r is zero or an integer from 1 to 3, indicating the number of consecutive serines in case r is 2 or 3;
q is an integer from 1 to 5, and
p is an integer from 1 to 10.
   In a specific embodiment of the recombinant KalbTGase substrate, t is 2 or 3. In another specific embodiment of the recombinant KalbTGase substrate, r is 1. In yet another specific embodiment of the recombinant KalbTGase substrate, p is any of 2 to 5, and in another specific embodiment p is 3 or 2.

In an embodiment of the recombinant KalbTGase substrate, T and each R^{m} are flexible linker amino acid sequences. In a specific embodiment of the recombinant KalbTGase substrate, at least one flexible linker amino acid sequence of T and/or R^{m} is selected from an amino acid sequence of the group consisting of

[[G₃]-[S]₁]₄-[G]₃ (Formula IIIa),

[[G₃]-[S]₁]₃-[G]₃ (Formula IIIb), and

[[G₃]-[S]₁]₂-[G]₃ (Formula IIIc).

In an embodiment of the recombinant KalbTGase substrate, at least one Lⁿ is selected from the group consisting of SEQ ID NO: 12, SEQ ID NO: 13, SEQ ID NO: 14, SEQ ID NO: 15, SEQ ID NO: 16, and SEQ ID NO: 17.

In an embodiment of the recombinant KalbTGase substrate, at least one rigid linker amino acid sequence of T and/or any of R^{m} is an independently selected amino acid sequence of Formula IV

[E-[A-]vK]w (Formula IV),
wherein
v is an integer from 2 to 5, and
w is an integer from 1 to 6.

In a specific embodiment of the recombinant KalbTGase substrate, v is 3. In another specific embodiment of the recombinant KalbTGase substrate, w is 2 to 5, and in another specific embodiment w is 3. In yet another specific embodiment of the recombinant KalbTGase substrate, the at least one rigid linker amino acid sequence of T or any of R^{m} is the amino acid sequence of Formula IVa

[E-[A-]₃K]₃ (Formula IVa).

In a specific embodiment of the recombinant KalbTGase substrate, at least one Lⁿ is selected from the group consisting of SEQ ID NO: 70, SEQ ID NO: 71, SEQ ID NO: 73, SEQ ID NO: 74, SEQ ID NO: 75, and SEQ ID NO: 76.

In a second aspect the present report provides a method of forming a target polypeptide with a covalently attached label, the method comprising the steps of
a. providing a KalbTGase substrate according to the first aspect and all its embodiments herein, the KalbTGase substrate comprising the target polypeptide and one or more acyl donor glutamine residue(s) for KalbTGase transglutaminase activity;
b. providing a label conjugate, wherein in the label conjugate the label is covalently attached to a Ktag, wherein the Ktag is a KalbTG lysine-containing-acceptor-motif or a functional analog thereof, wherein the Ktag comprises a primary amine group capable of being reacted with an acyl donor glutamine residue for KalbTGase transglutaminase activity in the presence of KalbTGase;
c. contacting the fusion polypeptide of (a) and the conjugate of the label of (b) with KalbTGase under conditions allowing transglutaminase activity to occur;
   thereby reacting an acyl donor glutamine residue with a primary amine group to form a covalent connection, and thereby forming the target polypeptide with the covalently attached label.

In a third aspect the present report provides a method of forming a target polypeptide with a covalently attached capture group, the method comprising the steps of
a. providing a KalbTGase substrate according to the first aspect and all its embodiments herein, the KalbTGase substrate comprising the target polypeptide and one or more acyl donor glutamine residue(s) for KalbTGase transglutaminase activity;
b. providing a capture group conjugate, wherein in the capture group conjugate the capture group is covalently attached to a Ktag, wherein the Ktag is a KalbTG lysine-containing-acceptor-motif or a functional analog thereof, wherein the Ktag comprises a primary amine group capable of being reacted with an acyl donor glutamine residue for KalbTGase transglutaminase activity in the presence of KalbTGase;
c. contacting the fusion polypeptide of (a) and the conjugate of the capture group of (b) with KalbTGase under conditions allowing transglutaminase activity to occur;
   thereby reacting an acyl donor glutamine residue with a primary amine group to form a covalent connection, thereby forming the target polypeptide with the covalently attached capture group.

In an embodiment of a method of forming a target polypeptide with a covalently attached label or capture group, the KalbTGase is the polypeptide of SEQ ID NO: 65.

In a fourth aspect the present report provides a labeled target polypeptide, obtained or obtainable by the method according to the second aspect and all its embodiments herein.

In a fifth aspect the present report provides a target polypeptide with a covalently attached capture group, obtained or obtainable by the method according to the third aspect and all its embodiments herein.

In a sixth aspect the present report provides a composition suitable for detecting in an isolated sample target antibodies specific for an antigen amino acid sequence, wherein the composition comprises a labeled target polypeptide according to the fourth aspect and all its embodiments herein, wherein the antigen amino acid sequence is comprised in the labeled target polypeptide. In an embodiment, the label is selected from the group consisting of a fluorescent dye, a chemiluminescent label, an iridium-containing electrochemiluminescent label, a ruthenium-containing electrochemiluminescent label, a single-stranded oligonucleotide or analog thereof, and a radiolabel. In a more specific embodiment the single-stranded oligonucleotide analog consists of L-LNA monomers known to the art from e.g. WO2019243391 and WO2020245377. In yet another more specific embodiment the label comprises Tris(2,2′-Bipyridyl) Ruthenium(II) Ion. Other examples of suitable labels for the purpose of the present report are disclosed in WO2017153574.

In an embodiment of the composition suitable for detecting in an isolated sample target antibodies specific for an antigen amino acid sequence, the composition further comprises a separate molecule comprising the unlabeled antigen amino acid sequence or a molecular mimic thereof, the separate molecule being attached to a capture group. In a specific embodiment, the separate molecule is a target polypeptide with a covalently attached capture group according to the fifth aspect the present report. In yet another specific embodiment, the capture group is selected from the group consisting of a hapten, digoxygenin and biotin.

In a seventh aspect the present report provides a method for detecting in an isolated sample a target antibody (═X) specific for an antigen amino acid sequence (═Y), said method comprising
a. forming an immunoreaction mixture by admixing a body fluid sample suspected of containing X with a labeled target polypeptide according to the fourth aspect and all its embodiments herein, wherein Y is comprised in the labeled target polypeptide,
b. maintaining the immunoreaction admixture of step (a) for a time period sufficient for allowing X present in the body fluid sample to immunoreact with Y comprised in the labeled target polypeptide to form an immunoreaction product (═X:Y) in the admixture; and
c. detecting the presence and/or the concentration of X:Y formed in step (b);
   thereby detecting in the isolated sample target antibody specific for the antigen amino acid sequence.

In an embodiment of the method for detecting in an isolated sample a target antibody (═X) specific for an antigen amino acid sequence (═Y), step (c) comprises the steps of
(a) contacting the immunoreaction admixture of step (a) or the admixture with immunoreaction product of step (b) with a separate molecule (═Z), wherein Z comprises a capture group and the unlabeled antigen amino acid sequence or a molecular mimic thereof;
(b) maintaining the admixture of step (i) for a time period sufficient for allowing an immunoreaction product to form in which X simultaneously binds Y and Z (═Z:X:Y);
(c) capturing Z:X:Y and separating Z:X:Y from the admixture of step (ii); and
(d) detecting the label present in captured Z:X:Y.

In an embodiment of the method for detecting in an isolated sample a target antibody (═X) specific for an antigen amino acid sequence (═Y), the molecular mimic of the antigen amino acid sequence is an anti-idiotypic antibody or an aptamer capable of forming an immunoreactant with the target antibody, wherein the antigen is capable of competing against the aptamer or anti-idiotypic antibody for target antibody binding. In a specific embodiment, the molecular mimic is an antibody-binding fragment of the anti-idiotypic antibody.

In another embodiment of the method for detecting in an isolated sample a target antibody (═X) specific for an antigen amino acid sequence (═Y), Z is a target polypeptide with a covalently attached capture group according to the fifth aspect the present report.

In yet another step (iii) comprises capturing Z:X:Y on a solid phase, and separating the solid phase with captured Z:X:Y from the admixture of step (ii). In a specific embodiment, the solid phase is a magnetic, paramagnetic or superparamagnetic bead, any of these known to the art. In yet a further specific embodiment, the surface of the solid phase comprises streptavidin, and Z comprises biotin as a capture group.

In an eighth aspect the present report provides the use of (i) a KalbTGase substrate according to the first aspect and all its embodiments herein, (ii) a label conjugate and (iii) KalbTGase for producing a labeled target polypeptide, wherein in the label conjugate the label is covalently attached to a Ktag, wherein the Ktag is a KalbTG lysine-containing-acceptor-motif or a functional analog thereof, wherein the Ktag comprises a primary amine group capable of being reacted with an acyl donor glutamine residue for KalbTGase transglutaminase activity in the presence of KalbTGase.

In a ninth aspect the present report provides the use of a labeled target polypeptide obtained or obtainable by the method according to the fourth aspect and all its embodiments herein for detecting in an isolated sample target antibodies specific for an antigen amino acid sequence.

In a tenth aspect the present report provides the use of a labeled target polypeptide according to the fourth aspect and all its embodiments herein and a separate target polypeptide with a covalently attached capture group according to the fifth aspect and all its embodiments herein for detecting in an isolated sample target antibodies specific for an antigen amino acid sequence.

In an eleventh aspect the present report provides a kit of parts for detecting in an isolated sample target antibodies specific for an antigen amino acid sequence, the kit containing a labeled target polypeptide according to the fourth aspect and all its embodiments herein, the labeled target polypeptide comprising the antigen amino acid sequence.

In an embodiment of the kit of parts for detecting in an isolated sample target antibodies specific for an antigen amino acid sequence, kit further contains a separate molecule which comprises a capture group and the unlabeled antigen amino acid sequence or a molecular mimic thereof. In an embodiment, the molecular mimic of the antigen amino acid sequence is an anti-idiotypic antibody or an aptamer capable of forming an immunoreactant with the target antibody, wherein the antigen is capable of competing against the aptamer or anti-idiotypic antibody for target antibody binding. In a specific embodiment, the molecular mimic is an antibody-binding fragment of the anti-idiotypic antibody. In a further embodiment, the separate molecule is a target polypeptide with a covalently attached capture group according to the fifth aspect herein.

In another embodiment of the kit of parts for detecting in an isolated sample target antibodies specific for an antigen amino acid sequence, the kit further contains a solid phase capable of capturing the capture group. In a more specific embodiment the solid phase is a magnetic, paramagnetic or superparamagnetic bead, any of these known to the art. In yet a further specific embodiment, the surface of the solid phase surface comprises streptavidin, and the capture group is biotin.

In a twelfth aspect the present report provides a DNA encoding a fusion polypeptide comprising a target polypeptide and one or more acyl donor glutamine residue(s) for KalbTGase transglutaminase activity, wherein a codon for N-terminal methionine is appended to a nucleotide sequence encoding the amino acid sequence of the KalbTGase substrate according to the first aspect and all its embodiments herein.

In a thirteenth aspect the present report provides an expression vector for recombinant expression in a transformed organism, the expression vector comprising the DNA according to the twelfth aspect and all its embodiments herein.

In a fourteenth aspect the present report provides a prokaryotic host organism stably transformed with an expression vector according to the thirteenth aspect and all its embodiments herein, wherein the transformed host organism is capable of expressing the encoded KalbTGase substrate from the expression vector

In a fifteenth aspect the present report provides a method of producing a recombinant KalbTGase substrate, the method comprising the steps of
(a) providing and culturing the transformed host organism according to the fourteenth aspect and all its embodiments herein,
(b) expressing in the transformed host organism of (a) the recombinant KalbTGase substrate, and
(c) purifying the expressed recombinant KalbTGase substrate from the transformed host organism,
thereby producing the recombinant KalbTGase substrate.

### EXAMPLES

### Example 1: Expression and Purification of Recombinant HIV1 gp41 and 6hel Antigens

Small-Scale Preparation of Recombinant 6hel Antigens for High Throughput Screening

242 Plasmids containing gp41-6hel genes with different point mutations and a C-terminal hexahistidine-tag were synthesized at Twist Bioscience and cloned into pET29a via the NdeI (5′-end) and XhoI (3′-end) restriction sites.

Small Scale Expression of Recombinant gp41-6hel Protein

After diluting the plasmids in 10 mM Tris-HCl buffer (pH 8.5) to 5 to 10 ng/μl, 1 μl of the DNA was added to HT96 BL21 (DE3) competent cell plates (Novagen). The transformation was done according to the manufacturer protocol and twenty microliter of the transformation reaction plated on 48-well LB-Kanamycin (50 μg/ml) agar plates (Teknova).

The cultivation and expression of all variants was performed in 96-well plate format. For the pre-culture one colony per mutant was picked in 96-well flat bottom micro titer plates (Corning) filled with 200 μl 4× Yeast-Kanamycin (50 μg/ml) medium per well. In addition, each plate contained at least one wild type gp41 antigen as reference. The cells were grown at 37° C. overnight without shaking. For long-term storage 50 μl of 50% (v/v) Glycerol were added before freezing. The expression was done in 96 deep-well plates including 1000 μl 4× Yeast-Kanamycin (50 μg/ml) medium with 0.1 mM IPTG per well. The mutants were expressed at 30° C. and 800 rpm (Microplate Shaker TiMix; Edmund Buhler GmbH) and harvested after 16 h by centrifugation at 4700 rpm for 10 min.

Small Scale Purification of Recombinant gp41-6hel Protein

For small scale purification of 6hel antigens, the bacterial cell pellets from 1 ml E. coli culture were lysed with 125 μl 100% BugBuster® (Merck Millipore) according to the manufacturer protocol. After adding 125 μl 2× equilibration buffer (0.1 M NaH2PO4 pH 8.0; 1% (v/v) Tween-20; 1 M NaCl; 40 mM Imidazole) and clearance of the cell lysates by centrifugation (4700 rpm, 10 min), the lysates were transferred to 96-well V bottom plates (Corning) using a pipetting robot (Biomek). Small scale purification was carried out with robotic pipet tips called PhyTips (PhyNexus) which are prepacked with Ni-NTA resins. At first, the Phytips were equilibrated with equilibration buffer (0.05 M NaH2PO4 pH 8.0; 0.5% (v/v) Tween-20; 0.5 M NaCl; 20 mM Imidazole). Then they were transferred to the samples for protein. binding In order to remove non-specific bound proteins, the Phytips were washed twice with washing buffer 1 (0.05 M NaH2PO4 pH 8.0; 0.5% (v/v) Tween-20; 0.5 M NaCl; 20 mM Imidazole), followed by two washing steps with washing buffer 2 (0.05 M NaH2PO4 pH 8.0; 0.5% (v/v) Tween-20; 0.15 M NaCl; 20 mM Imidazole). Finally, the 6hel antigens were eluted in 100 μl elution buffer (0.05 M NaH2PO4 pH 8.0; 0.5% (v/v) Tween-20; 0.15 M NaCl; 200 mM Imidazole). Protein samples were analyzed by SDS-PAGE gel. After Ni-NTA purification, a buffer exchange to conjugation buffer (0.15 M KH2PO4 pH 8.0; 0.1 M KCl; 0.5 mM EDTA) was conducted using Pierce™ 96-well micro-dialysis plates according to the instructions provided by Pierce Biotechnology.

Small Scale Ruthenylation and Biotinylation of Recombinant gp41-6hel Protein

Conjugation was performed in black 96-well half area plates (Corning) using NHS-chemistry. Prior to conjugation protein concentration in each well was determined in micro titer plates by BCA assay using the Pierce™ BCA Protein Assay Kit (ThermoFisher). Per well 180 μl of BCA solution were added to 20 μl of purified antigen and measured at 562 nm with a Tecan Sunrise™ microplate reader.

Antigen (approx. 1 mg/ml) and label were rapidly mixed to a final antigen to label ratio of one to four for ruthenium conjugation and one to five for biotin conjugation and a DMSO concentration of 10% (v/v). The plates were incubated at room temperature for 30 min at 600 rpm. The labeling reaction was stopped by adding L-lysine to a final concentration of 10 μM. For small-scale preparations free unbound ruthenium label were not removed, while free unbound biotin label was removed by usage of PD MultiTrap™ G-25 96-well plates (GE Healthcare). Concentrations of ruthenylated and biotinylated antigens were determined by usage of BCA assay as described above. The ruthenylated and biotinylated gp41-6hel mutants were stored at 4° C. until the assessment by the Elecsys test system.

In summary, 171 (71%) out of the 242 6hel mutations could be successfully purified, labeled and further assessed via immunoassay. The missing 71 variants, either failed DNA synthesis, could not be expressed or the yield of purified protein was too low to perform a labeling reaction.

Large-Scale Preparation of Recombinant HIV1 gp41 and 6hel Antigens for Thorough Screening

Plasmids containing recombinant HIV1 gp41(aa536-681) and 6hel genes with different point mutations and a C-terminal hexahistidine-tag were synthesized at Eurofins Genomics GmbH and cloned into pET24a(+) via the NdeI (5′-end) and XhoI (3′-end) restriction sites.

Furthermore, recombinant gp41(aa536-681) was N-terminally fused to two SlyD chaperones from E. coli via a Glycine-Serine rich linker (Scholz, C. et al., J. Mol. Biol. (2005) 345, 1229-1241) resulting in the EcSlyD-EcSlyD-gp41 fusion protein in the following only referred to as gp41.

Expression of gp41 as well as 6hel constructs was performed in BLR(DE3) E. coli cells using standard LB medium and IPTG induction for three hours at 37° C. Cells were harvested by centrifugation (20 min, 5000 g) and stored at −20° C. upon further processing.

Large Scale Purification of Recombinant HIV1 gp41 and 6hel Antigens

Recombinant HIV1 gp41 and 6hel antigens were purified under denaturing conditions followed by an on-column renaturation. In detail, bacterial pellets from 700 ml E. coli culture were resuspended in chaotropic lysis buffer (50 mM sodium phosphate pH 8.0; 4 M guanidinium chloride; 5 mM imidazole) and stirred at room temperature for 90 min. For clearance the cell lysate was centrifuged and filtered (5/0.8/0.2 μm). Clarified supernatant was applied to a Roche cOmplete His-tag purification column equilibrated with lysis-buffer. Unspecifically bound proteins were removed from the column by a thorough wash with lysis-buffer to baseline. Refolding of antigens was performed by on-column renaturation using refolding-buffer (50 mM sodium phosphate pH 8.0; 100 mM NaCl). Refolded target protein was eluted from the column with imidazole containing elution buffer (50 mM sodium phosphate pH 8.0; 50 mM imidazole; 100 mM NaCl). For buffer exchange and polishing, the protein was applied to a Superdex 200 column equilibrated with SEC-buffer1 (50 mM Tris-HCl pH 8.0; 150 mM KCl) for site specific labeling or SEC-buffer2 (150 mM potassium phosphate pH 8.9; 100 mM KCl; 0.5 mM EDTA) for labeling using NHS-chemistry. Gp41 elutes in three peaks with one prominent peak representing an oligomeric arrangement. The oligomeric fraction was concentrated and processed to biotinylation and ruthenylation. 6hel elutes in one peak, which was concentrated and processed to biotinylation and ruthenylation

### Example 2: Ruthenylation and Biotinylation of Recombinant gp41 Protein

Large Scale Ruthenylation and Biotinylation of Recombinant HIV1 gp41 and 6hel Antigens Using NHS-Chemistry

For conjugation of the antigens with Biotin or Ruthenium protein concentration should be ideally 10 mg/ml in SEC-buffer 2. Conjugation was performed with a molar antigen to label ratio of one to four and a DMSO concentration of 5% (v/v) using NSH-chemistry. Label and antigen were rapidly mixed and stirred at room temperature for 30 min. The labeling reaction was stopped by adding L-lysine to a final concentration of 10 mM. For large scale preparations free unbound label was removed from the reaction by size exclusion chromatography using a Superdex 200 Increase (GE Healthcare) column equilibrated with storage-buffer (50 mM sodium phosphate pH 7.5; 100 mM KCl; 0.5 mM EDTA). Concentration of ruthenylated antigens was determined by the usage of BCA assay and concentration of biotinylated antigens was done by absorption measurement at 280 nm.

Large Scale Ruthenylation and Biotinylation of Recombinant HIV1 gp41 and 6hel Using Transglutaminase

Recombinant Transglutaminase from Kutzneria albida (KalbTG) can be used to site specifically label antigens by forming a Gln-Lys isopeptide bond between the Qtag containing antigen and the respective containing K-tag label (Steffen, W. et al. J. Mol. Biol. (2017) 292, 15622-1563). For conjugation of the HIV1 antigens with Biotin or Ruthenium the protein concentration should be ideally 10 mg/ml in SEC-buffer2. Conjugation was performed with a molar Qtag to label ratio of 1:5 and an enzyme to antigen dearth of 1:300. Antigen, label and activated enzyme were mixed and incubated for 20 hours at 37° C. while gentle mixing. After 20 hours of incubation, the reaction was stopped by adding 10 mM ammonium sulfate. Finally, free unbound label and KalbTG was removed from the labeled antigen by size exclusion chromatography using a Superdex 200 Increase (GE Healthcare) column equilibrated with storage-buffer (50 mM sodium phosphate pH 7.5; 100 mM KCl; 0.5 mM EDTA). Concentration of ruthenylated antigens was determined by the usage of BCA assay and concentration of biotinylated antigens was determined by absorption measurement at 280 nm.

Large Scale Biotinylation of Recombinant HIV1 gp41 and 6hel Using Sortase

Recombinant sortase can be used to site specifically label antigens by forming a peptide bond between the threonine of the C-terminal sortase recognition site (LPETG) and a glycine residue in the respective label. For conjugation of the HIV1 antigens with Biotin by sortase the protein concentration should be ideally, 10 mg/ml in phosphate free SEC-buffer1. Conjugation was performed in the presence of 10 mM calcium chloride with an antigen to label ratio of 1:50 and an enzyme input of 50 U per pmol antigen. Antigen, label and activated enzyme were mixed and incubated for 1 hour at 37° C. while gentle mixing. After 1 hours of incubation, the reaction was loaded on Roche cOmplete His-tag resin to remove sortase as well as unlabeled antigen from the reaction mix. Finally, free unbound label was removed by size exclusion chromatography using a Superdex 200 Increase (GE Healthcare) column equilibrated with storage-buffer (50 mM sodium phosphate pH 7.5; 100 mM KCl; 0.5 mM EDTA). Concentration of biotinylated antigens was determined by absorption measurement at 280 nm.

### Example 3: Biochemical Analysis of Recombinant HIV1 gp41 and 6hel Antigens

Spectroscopic Measurements of Recombinant HIV1 gp41 and 6hel Antigens

Protein concentration measurements were performed with a NanoDrop One® Micro-UV/Vis-spectrophotometer (Thermo Scientific). The molar extinction coefficients (F280 nm) of the antigens was calculated using the equation reported in Pace et al. (Protein Sci. 1995 November; 4(11):2411-23).

TABLE 1
Protein parameters of the five best recombinant HIV1 gp41 and 6hel antigens

| | | | | ε_{280 nm} |
|---|---|---|---|---|
| | number of | molecular | | [M − 1 |
| antigen variant | aa residues | weight [Da] | pI | cm−1] |
| 6hel-N(543-581)x3/C(625-662)x3-Srt | 269 | 30635.14 | 5.46 | 53970 |
| (SEQ ID NO: 82) | | | | |
| 6hel-N(543-581)x3/C(625-662, | 275 | 31064.47 | 5.03 | 56950 |
| N636D/H643Y)x2/C(625-662)x1-Srt | | | | |
| (SEQ ID NO: 83) | | | | |
| 6hel-N(543-581)x3/C(625-662, | 274 | 31273.72 | 5.10 | 61420 |
| N636D/H643Y)x3-Qtag | | | | |
| (SEQ ID NO: 84) | | | | |
| EcSlyD-EcSlyD-gp41((536-681; | 547 | 59100.63 | 4.84 | 86860 |
| N637E/H643Y)-Qtag | | | | |
| (SEQ ID NO: 85) | | | | |
| EcSlyD-EcSlyD-Qtag-gp41((536-681; | 599 | 63722.31 | 5.01 | 95800 |
| N637E/H643Y) | | | | |
| (SEQ ID NO: 86) | | | | |

Circular Dichroism (CD) Spectra of Recombinant HIV1 6hel Antigens

Far-UV CD spectra (190-250 nm) of 6hel antigens were recorded with a Jasco-720 spectropolarimeter and finally converted into the mean residue ellipticity (Θmrw,λ). All samples were diluted to 0.21 mg/ml in 50 mM potassium phosphate pH 7.5, 100 mM KCl, 0.5 mM EDTA. Adjustments at the spectrometer during measurement were as follow: 0.2 cm pathlength, scanning range of 190-330 nm, with a scanning speed of 20 nm/min, a bandwidth of 2.0 nm, a resolution of 0.5 nm and a response of 1 sec. All spectra were measured in nine repeats and averaged.

In the far-UV range CD spectroscopy allows to analyze the secondary structure proteins as absorption in this UV range is mainly caused by the peptide bond. Therefore, far-UV CD spectra of the all-helical 6hel antigen in comparison to the mutated variants give reliable insights into the structure of the antigen and the effect of the point mutations on protein folding.

HPLC Analysis of Recombinant HIV1 6hel Antigens

To analyse the purity and the aggregation tendency of the mutated antigens and also to estimate the molecular weight of the purified 6hel antigens HPLC analysis was performed. Therefore, at least 25 μg of the recombinant proteins was loaded onto a Superdex 200 column using 50 mM potassium phosphate pH 7.5, 100 mM KCl and 0.5 mM EDTA as mobile phase. As a reference, an internal HPLC standard was analyzed too. The HPLC analysis allows to assess the aggregation behavior of the mutated 6hel antigens in comparison to the wild type construct.

### Example 4: Immunological Reactivity of the Different Recombinant HIV1 gp41 and 6hel Antigens in an Anti-HIV Immunoassay

The immunological reactivity (antigenicity) of the HIV1 gp41 and 6hel variants was assessed in automated Elecsys® cobas analyzers (Roche Diagnostics GmbH) using the double antigen sandwich (DAGS) format. Signal detection in automated Elecsys® cobas analyzers is based on electrochemiluminescence. In case of a DAGS assay format the biotinylated capture-antigen is immobilized on the surface of a streptavidin coated magnetic bead whereas the same detection-antigen is conjugated with a ruthenium complex. Upon activation the ruthenium complex switches between the redox states 2+ and 3+ resulting in a light signal. In the presence of specific immunoglobulins, in this case anti-HIV IgG antibodies in human sera, the ruthenium complex is bridged to the solid phase and light emission at 620 nm is triggered at the electrode by adding tripropylamine.

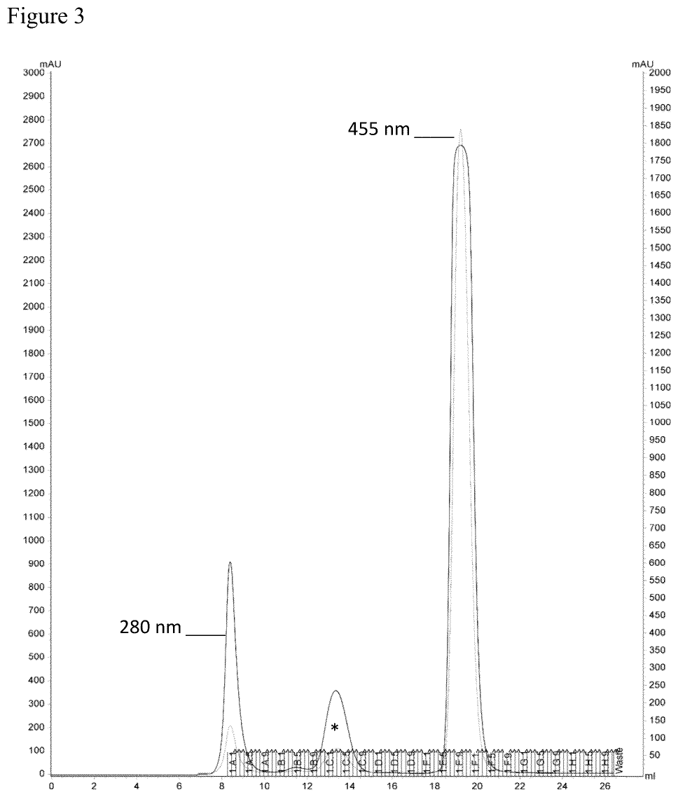

All 171 mutated variants of recombinant 6hel from small scale expression and labeling (FIG. 3) were examined in this study to evaluate their binding potential to anti-HIV1 IgG antibodies.

Immunological Reactivity of the Different Recombinant HIV1 gp41 and 6hel Antigens in a DAGS Assay Setup

For a more thorough analysis approximately the best 20 mutations in the 6hel antigen, which were identified in an initial screening to have an improved immunological specificity, were expressed and labeled in large scale and comprehensively analyzed in a DAGS assay setup. Furthermore, the same selected mutations were also transferred into the HIV1 gp41 antigen (WO03/000877) and their specificity evaluated. In addition to these constructs, 6hel as well as gp41 antigens containing combinations of the most promising mutations were produced and assessed.

In detail, the different gp41-biotin or 6hel-biotin and gp41-ruthenium or 6hel-ruthenium antigens were used in reagent buffer 1 (R1) and R2, respectively. Labeled recombinant gp41 antigens were used at concentrations between 30 ng/ml and 300 ng/ml in R1 and R2. The concentration of the various labeled 6hel antigens was between 2 ng/ml and 130 ng/ml in R1 and R2 dependent on the mutation.

To avoid immunological cross reactions via the chaperone fusion units of the recombinant HIV1 gp41 antigens unlabeled EcSkp-EcSlyD-EcSlyD (EP2893021(B1)) or chemically polymerized EcSlyD-EcSlyD were added in large excess (5-30 μg/ml) to the reaction buffer as anti-interference substances.

To assess the specificity and the sensitivity of the different recombinant HIV1 gp41 and 6hel antigens Elecsys measurements with HIV negative and positive as well as seroconversion samples were analyzed.

The results of three of the best antigens (SEQ ID Nos 1, 2 and 3) are shown in FIG. 6. In detail, A) Cut-off indices (COI) of ten highly positives HIV samples from patients infected with different HIV-1 subtypes. All samples tested with the improved anti-HIV module (AHIVII) are positive as already with the standard AHIVI module. COI values<1 are indicated as non-reactive, whereas samples with a COI>1 indicate the presence of anti-HIV antibodies. B) Comparison of the performance of the standard Elecsys HIV Duo assay (HIV Duo I in black) with the optimized Elecsys HIV Duo II assay (HIV Duo II in grey), as well as the separate comparison of the anti-HIV module AHIV I and AHIV II in grey and black, respectively. The comparison was performed on five commercially available seroconversion panels (1-5) with sequential blood draws. The optimized anti-HIV II module shows a higher sensitivity compared to the AHIV I module. The higher sensitivity of the AHIV II module particularly kicks in in the seroconversion panel two and three. In these two panels, blood draw nine or five (highlighted in grey) are negative in the AHIV I module and become clearly positive in the optimized AHIV II module. This higher sensitivity reduces the risk of the second window phase after infection and significantly decreases the risk of false negative HIV results. C) Graphical and D) Numerical representation of the specificity of the present and the optimized anti-HIV module in black and grey, respectively. Determination of the specificity of both modules was performed with 6046 HIV negative routine samples from different vendors. Setting the threshold of HIV positivity to a COI>1 (highlighted in bold) the standard AHIV I module showed four false positive samples resulting in a specificity of 99.92. Whereas the optimized AHIV II module does not show any signals with a COI>1, resulting in a specificity of 100. In summary, the here performed experiments showed a significant optimization in sensitivity as well as specificity of the AHIV II module compared to the AHIV module.

### Example 5: Data of an External Specificity Study

To thoroughly evaluate the specificity of the mutated and thus optimized gp41 and 6hel antigens 15,242 routine blood samples were analyzed in an external study (FIG. 7a). The assessment was performed by an independent laboratory using both the AHIV module of the Elecsys HIV Duo as well of the optimized Elecsys HIV Duo II assay (containing SEQ ID NO. 79, 80 and 81).

Within this study 44 samples resulted in false positive signals within the Elecsys HIV Duo I assay (specificity 99.71%) whereas only 7 false positive samples were detected using the optimized HIV Duo II assay (specificity 99.95%) (FIG. 7A). 21 false positive samples of the Elecsys HIV Duo I assay and 4 of the HIV Duo II are caused by the gp41 and 6hel antigens within the anti-HIV module of the HIV Duo Elecsys assays (FIG. 7b). Thus the significant specificity improvement of the mutated and optimized gp41 and 6hel antigens could also be shown within an independent external study and the interference potential of the gp41 antigen dropped to <20% after optimization.

### Example 6: Improvement of Sensitivity and Specificity of an HIV Immunoassay by Combinations of Mutated and Optimized HIV gp41 Antigens

In comparison to an HIV immunoassay containing HIV gp41 antigens comprising SEQ ID NO. 10, not only a combination of SEQ ID NO. 79, 80 and 81 show a significantly improved antigenicity but already a combination of two of the optimized HIV gp41 antigens (SEQ ID NO. 79 and 80 or SEQ ID NO. 79 and 81) show a significantly improved immunological reactivity (FIG. 8). The specificity of the different antigen combinations was assessed by the analysis of 103 HIV negative samples (FIG. 8a). When using the not further optimized gp41 antigen only 91 out of the 103 negative samples (88.35%) show a COI between 0.02 and 0.05, whereas at least 98% of the negative samples are within this COI range when using different combinations of the mutated gp41 and 6hel antigens (FIG. 8a column 2 to 3). Thus the scattering of the samples is significantly lower when using the optimized antigens. In addition, not only the specificity and the scattering is improved but also the sensitivity of the different combined optimized gp41 and 6hel antigens is significantly better (FIG. 8b). For examples sample Sero01 clearly shows that the sensitivity is much higher in all combinations using the mutated antigens, with being SEQ ID NO. 79, 80 and 81 the best combination.

### Example 7: Ruthenylation of a Fusion Polypeptide Containing Four YRYRQ Qtags

Recombinantly produced fusion polypeptides containing Qtags were labeled with Ruthenium label, wherein the Ruthenium label was provided as a conjugate with a Ktag peptide (GRYESKG, SEQ ID NO: 95), the conjugate comprising an alkyl amine donor group of the lysine residue for KalbTGase. In separate labeling reactions, the fusion polypeptides which contained Qtags, were mixed with the Ru conjugate comprising a Ktag. KalbTGase was added and incubated. Conditions were as described previously by Steffen et al. (2017) J Biol Chem 292, 15622-15635. Unreacted label-Ktag conjugate was separated from labeled fusion polypeptides by way of size exclusion chromatography. Elution was monitored by determining optical density at 280 nm and at 455 nm wavelengths.

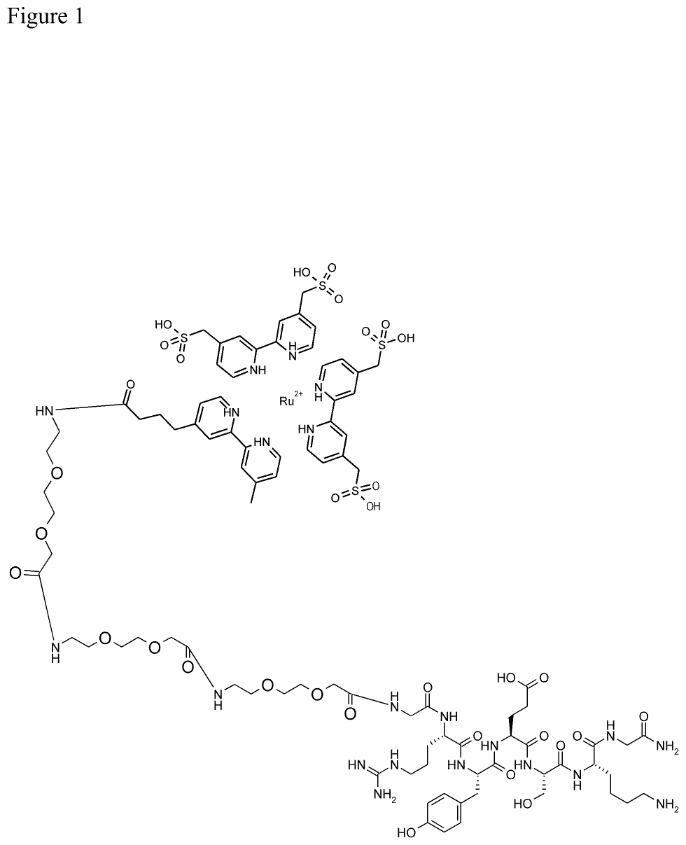

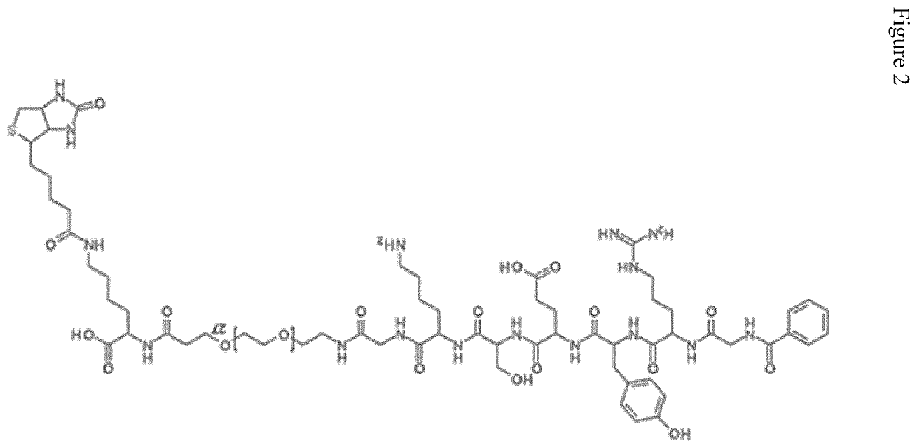

In the same way, any fusion polypeptide with Qtags was labeled. Exemplary Ru label is disclosed on FIG. 1. For biotin-Ktag attachment suitable compounds are disclosed on FIG. 2. Attachment of biotin-Ktag using KalbTGase is performed similarly as provided above, with biotin-Ktag replacing the Ru label.

### Example 8: Use in an Immunoassay

The ruthenylated fusion polypeptide of SEQ ID NO:96 labeled as described in Example 7 was used in an immunoassay for detecting in a sample a target antibody (═X) specific for an antigen amino acid sequence (═Y), said method comprising
(a) forming an immunoreaction mixture by admixing a serum or plasma sample with or without X with a labeled target polypeptide, wherein a dengue virus NS1 antigen is comprised in the labeled target polypeptide,
(b) maintaining the immunoreaction admixture of step (a) for a time period sufficient for allowing X present in the body fluid sample to immunoreact with Y comprised in the labeled target polypeptide to form an immunoreaction product (═X:Y) in the admixture; and
(c) performing the steps of
(d) contacting the immunoreaction admixture of step (a) or the admixture with immunoreaction product of step (b) with a separate molecule (═Z), wherein Z comprises a biotin as a capture group and the unlabeled antigen amino acid sequence;
   i. maintaining the admixture of step (d) for a time period sufficient for allowing an immunoreaction product to form in which X simultaneously binds Y and Z (═Z:X:Y);
   ii. capturing Z:X:Y on a magnetic bead which is coated on its surface with streptavidin, and magnetically separating Z:X:Y from the admixture of step (ii); and
   iii. detecting the label present in captured Z:X:Y immobilized on the magnetic bead.

The detection assay was performed on an ELECSYS® analyzer capable of detecting immobilized Ru-label by means of electrochemoluminescence (ESL). Parallel experiments were made using conventionally prepared Ru-labeled fusion polypeptide, however without Qtags. These molecules were labeled using a crosslinking reaction. Samples were provided which were known to be positive or negative for antibodies specific for Dengue virus NS1 antigen. Table 2 summarizes the data.

Double-antigen sandwich principle, typical duration of assay: 18 minutes.
1st incubation: defined volumes of sample, biotinylated recombinant antigen and the same recombinant antigen labeled with a ruthenium complex form a sandwich complex.
2nd incubation: After addition of streptavidin-coated microparticles, the complex becomes bound to the solid phase via interaction of biotin and streptavidin.

The reaction mixture is aspirated into the measuring cell where the microparticles are magnetically captured onto the surface of the electrode. Unbound substances are then removed with a specific buffer that at the same time provides the reagent for light emission.

Application of a voltage to the electrode then induces chemiluminescent emission which is measured by a photomultiplier.

TABLE 2
ESL light counts

| | | chemically | chemically |
|---|---|---|---|
| Control sample | KalbTGase- | labeled, | labeled, |
| type | labeled | experiment 1 | experiment 1 |
| Negative 1 | 901 | 2438 | 2323 |
| Negative 2 | 889 | 2068 | 2043 |
| Positive 1 | 7172 | 7303 | 6660 |
| Positive 2 | 10376 | 7665 | 7743 |
| Positive 3 | 4190 | 3510 | 3772 |
| Positive 4 | 16039 | 15978 | 13912 |

It becomes evident from the data of Table 2 that the baseline signal generated by the negative samples is much lower in the case of KalbTGase-labeled fusion polypeptide than in the experiments with chemically labeled fusion polypeptide.

### Example 9: Use in an Immunoassay, Different Temperature Regimes

When Example 8 was repeated after incubating the labeled fusion polypeptides at 4° C. and 35° C. for one week, the effect could be reproduced. This points to increased stability of KalbTGase-labeled fusion polypeptide.

TABLE 3a
ESL light counts, omparison of detection assay reagents
stored for 1 week at 4° C. and 35° C.

| | | |
|---|---|---|
| | KalbTGase-labeled, | |
| Control | experiment | chemically labeled |

| sample type | 4° C. | 35° C. | 4° C. | 35° C. |
|---|---|---|---|---|
| Negative 1 | 1243 | 1193 | 1466 | 1131 |
| Negative 2 | 1185 | 1145 | 1135 | 953 |
| Positive 1 | 10163 | 10013 | 5416 | 4605 |
| Positive 2 | 10426 | 9772 | 4987 | 4111 |
| Positive 3 | 20447 | 20737 | 9256 | 7925 |

TABLE 3b
relative amounts of light counts after 35° C. incubation,
in relation to 4° C. incubation (100%)

| | | |
|---|---|---|
| | KalbTGase-labeled, | |
| Control | experiment | chemically labeled |

| | | | | |
|---|---|---|---|---|
| sample type | 4° C. | 35° C. | 4° C. | 35° C. |

| | | |
|---|---|---|
| Negative 1 | 96% | 77% |
| Negative 2 | 97% | 84% |
| Positive 1 | 99% | 85% |
| Positive 2 | 94% | 82% |
| Positive 3 | 101% | 86% |

From the data it can be seen that not only the light counts from the negative control samples but also for the positive controls remained very stable when the fusion polypeptide was labeled using Qtags, Ktag-Ru label and KalbTGase.

### Example 10: Use in an Immunoassay

Example 8 was repeated in a modified form with a different antigen, i.e. instead of the Dengue virus NS1 antigen HIV gp41 was used. The fusion polypeptide was that of SEQ ID NO: 86. The ruthenylated fusion polypeptide of SEQ ID NO:86 labeled as described in Example 7 was used in an immunoassay for detecting in a sample a target antibody (═X) specific for an antigen amino acid sequence (═Y), said method comprising
(a) forming an immunoreaction mixture by admixing a serum or plasma sample with or without X with a labeled target polypeptide, wherein a HIV gp41 antigen is comprised in the labeled target polypeptide,
(b) maintaining the immunoreaction admixture of step (a) for a time period sufficient for allowing X present in the body fluid sample to immunoreact with Y comprised in the labeled target polypeptide to form an immunoreaction product (═X:Y) in the admixture; and
(c) performing the steps of
(d) contacting the immunoreaction admixture of step (a) or the admixture with immunoreaction product of step (b) with a separate molecule (═Z), wherein Z comprises a biotin as a capture group and the unlabeled antigen amino acid sequence;
   i. maintaining the admixture of step (d) for a time period sufficient for allowing an immunoreaction product to form in which X simultaneously binds Y and Z (═Z:X:Y);
   ii. capturing Z:X:Y on a magnetic bead which is coated on its surface with streptavidin, and magnetically separating Z:X:Y from the admixture of step (ii); and
   iii. detecting the label present in captured Z:X:Y immobilized on the magnetic bead.

Further variations were introduced by attaching biotin to Z either by chemical coupling or by KalbTGase-mediated labeling.

The detection assay was performed on an ELECSYS® analyzer capable of detecting immobilized Ru-label by means of electrochemoluminescence (ESL).

TABLE 4
Comparison of Ruthenium labeling and biotin labeling
using chemical labeling or KalbTGase labeling,
Fusion polypeptide was always 70 ng/ml

| Biotin labeling | | | |
|---|---|---|---|
| chemical | KalbTGase | chemical | KalbTGase |
| Ru-labeling | | | |
| chemical | chemical | KalbTGase | KalbTGase |

ELECSYS ® light counts

| | | | | |
|---|---|---|---|---|
| Negative | 559 | 559 | 565 | 571 |
| calibrator | | | | |
| Positive | 4858 | 6339 | 6546 | 8352 |
| calibrator | | | | |
| Sample 1 | 1254 | 1786 | 1504 | 2208 |
| Sample 2 | 6252 | 9074 | 7949 | 11471 |
| Sample 3 | 2823 | 3802 | 3503 | 4766 |
| Sample 4 | 2728 | 3861 | 3079 | 4507 |
| Sample 5 | 6667 | 10344 | 8036 | 12509 |
| Sample 6 | 20398 | 29386 | 24919 | 35582 |
| Sample 7 | 3139 | 4632 | 3752 | 5586 |
| Sample 8 | 1127 | 1873 | 1348 | 2318 |
| Sample 9 | 14605 | 20671 | 17413 | 24979 |
| Sample 10 | 3485 | 4971 | 4304 | 5989 |

Relative amounts, in %

| | | | | |
|---|---|---|---|---|
| Negative | 100 | 100 | 101 | 102 |
| calibrator | | | | |
| Positive | 100 | 130 | 135 | 172 |
| calibrator | | | | |
| Sample 1 | 100 | 142 | 120 | 176 |
| Sample 2 | 100 | 145 | 127 | 183 |
| Sample 3 | 100 | 135 | 124 | 169 |
| Sample 4 | 100 | 142 | 113 | 165 |
| Sample 5 | 100 | 155 | 121 | 188 |
| Sample 6 | 100 | 144 | 122 | 174 |
| Sample 7 | 100 | 148 | 120 | 178 |
| Sample 8 | 100 | 166 | 120 | 206 |
| Sample 9 | 100 | 142 | 119 | 171 |
| Sample 10 | 100 | 143 | 124 | 172 |

From the table it becomes clear that KalbTGase-based labeling leads to best results, not only for attaching Ruthenium label but also for attaching biotin label to fusion polypeptides with Qtags.

### Example 10: Use in an Immunoassay

The fusion polypeptides of SEQ TD NOs: 91, 92, 93, 94 and 96 were separately labeled with Ruthenium using KalbTGase. The fusion polypeptides with the capture group were biotinylated chemically. The ruthenylated fusion polypeptides differed in their linker amino acid sequences and in the Qtags used. All labeled polypeptides were compared with respect to the signals they generated in the immunoassay to detect antibodies. Z:X:Y sandwich complexes were immobilized and ELECSYS® light counts were measured.

TABLE 5
Signal yield with fusion polypeptides of SEQ ID NOs: 91, 92, 93, 94
and 96 Biotin label density in Z was the same for each experiment

| | | SEQ ID | | SEQ ID | |
|---|---|---|---|---|---|
| | SEQ ID | NO: 92, | SEQ ID | NO: 94, | SEQ ID |
| | NO: 91, | shortened | NO: 93, | flexible and | NO: 96, |
| | flexible | flexible | rigid | rigid, | flexible |
| Fusion | linker, Qtag is | linker, Qtag is | linker, Qtag is | linkers Qtag is | linker, Qtag is |
| polypeptide | YRYRQ | YRYRQ | YRYRQ | YRYRQ | RVRQR |
| Biotin | 1 | 1 | 1 | 1 | 1 |
| incorporation | | | | | |
| Average | 3.71 | 2.3 | 0.9 | 1 | 1.3 |
| Ruthenium | | | | | |
| label | | | | | |
| incorporation | | | | | |
| per molecule | | | | | |
| Negative | 672 | 692 | 643 | 636 | 686 |
| control 1 | | | | | |
| Positive | 3419 | 4328 | 1704 | 1728 | 2405 |
| control 1 | | | | | |
| Negative | 667 | 696 | 640 | 613 | 688 |
| control 2 | | | | | |
| Positive | 2530 | 3105 | 1360 | 1356 | 1840 |
| control 2 | | | | | |
| Sample 1 | 11380 | 20080 | 3802 | 5025 | 7686 |
| Sample 2 | 26536 | 33982 | 11407 | 10937 | 17295 |
| Sample 3 | 2062 | 3402 | 1114 | 1257 | 1663 |
| Sample 4 | 42143 | 55189 | 18059 | 17658 | 28254 |
| Sample 5 | 889 | 961 | 724 | 705 | 815 |
| Sample 6 | 3118 | 3868 | 1487 | 1568 | 2142 |
| Sample 7 | 937 | 1114 | 721 | 730 | 845 |
| Sample 8 | 918 | 878 | 704 | 684 | 888 |
| Sample 9 | 970 | 1112 | 766 | 752 | 831 |
| Sample 10 | 1561 | 1778 | 937 | 983 | 1235 |
| Sample 11 | 667 | 677 | 646 | 630 | 687 |

### Example 11: Quantification of Ru Label Incorporation

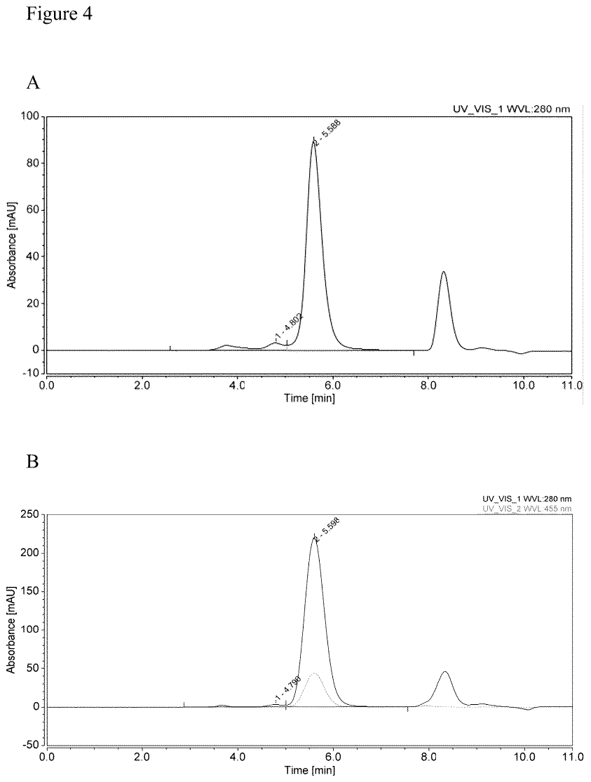

The products obtained from the KalbTGase incubation as described in Example 7 were subjected to size exclusion chromatography using a Superdex® 200 column. Elution was monitored by determining absorption at 455 nm and 280 nm wavelengths. Exemplary elution profiles are shown in FIG. 3. Fractions under the peak marked by an asterisk were pooled. Protein concentration was determined using a BCA assay. Using the extinction coefficient of Ru label alone as reference, the molar ratio of Ru label per labeled fusion polypeptide was calculated. Typically ratios between 0.5 and 4 were found, depending on accessibility of Qtags for the labeling reaction in the presence of KalbTGase. An exemplary chromatogram of a purified Ru-labeled fusion polypeptide is shown in FIG. 4, together with a further chromatogram of a fusion polypeptide with biotin attached to Qtags.

1. A recombinant KalbTGase substrate comprising a fusion polypeptide of Formula I,
_{N-terminus}[Aⁿ-Lⁿ-]ₙB_{C-terminus} (Formula I),
wherein
a hyphen “-” denotes a peptide bond;
n is an integer number from 1 to 6;
a pair of square brackets “[” and “]” delimits N-terminal and C-terminal borders of an amino acid sequence which is present n times;
Aⁿ is a FKBP chaperone amino acid sequence, and in case of n>1, each Aⁿ is an independently selected FKBP chaperone amino acid sequence;
Lⁿ is a coupling amino acid sequence, and in case of n>1, each Lⁿ is an independently selected coupling amino acid sequence,
B is a target polypeptide amino acid sequence;
and wherein
each Lⁿ is of Formula IIa or Formula IIb
T[-Qtag^{m}-R^{m}]ₘ (Formula IIa),
[R^{m}-Qtag^{m}-]ₘT (Formula IIb);
wherein
m is an integer number from 1 to 10;
a pair of square brackets “[” and “]” delimits N-terminal and C-terminal borders of an amino acid sequence which is present m times;
T is a flexible or rigid linker amino acid sequence comprising 5 to 500 amino acids;
R^{m} is a flexible or rigid linker amino acid sequences comprising 5 to 500 amino acids, and in case of m>1, each R^{m} is an independently selected flexible or rigid linker amino acid sequences comprising 5 to 500 amino acids;
Qtag^{m} is an amino acid sequence motif containing an acyl donor glutamine residue for KalbTGase transglutaminase activity, and in case of m>1, each Qtag^{m} is an independently selected amino acid sequence motif containing an acyl donor glutamine residue for KalbTGase transglutaminase activity;
with the proviso that SEQ ID NO: 18 and SEQ ID NO: 19 are excluded from Formula I.

2. The recombinant KalbTGase substrate of claim 1, wherein Aⁿ is selected from the group consisting of SEQ ID NO: 4, SEQ ID NO: 5, and SEQ ID NO: 6, or a functional variant thereof with a sequence identity of 85% or higher.

3. The recombinant KalbTGase substrate of claim 1, wherein n is 2 to 6.

4. The recombinant KalbTGase substrate of claim 3, wherein each Aⁿ is independently selected from the group consisting of SEQ ID NO: 4, SEQ ID NO: 5, and SEQ ID NO: 6, or a functional variant thereof with a sequence identity of 85% or higher.

5. The recombinant KalbTGase substrate of claim 3, wherein all Aⁿ are derived from a single member of the group consisting of SEQ ID NO: 4, SEQ ID NO: 5, and SEQ ID NO: 6, and the Aⁿ amino acid sequences are at least 90% identical.

6. The recombinant KalbTGase substrate of claim 3, wherein each Aⁿ amino acid sequence functionally preserves FKBP chaperone activity in the fusion polypeptide.

7. The recombinant KalbTGase substrate of claim 1, wherein B is an antigen amino acid sequence.

8. The recombinant KalbTGase substrate of claim 7, wherein B comprises the amino acid sequence of SEQ ID NO: 76 or SEQ ID NO: 77.

9. The recombinant KalbTGase substrate of claim 1, wherein Qtag^{m} is selected from YRYRQ (SEQ ID NO: 20) and RVRQR (SEQ ID NO: 21), or in case of m>1, each Qtag^{m} is independently selected from the group consisting of YRYRQ (SEQ ID NO: 20) and RVRQR (SEQ ID NO: 21).

10. The recombinant KalbTGase substrate of claim 1, wherein R^{m} and T are selected from
(i) a flexible linker amino acid sequence composed of glycine (G) and optionally serine (S),
and
(ii) a rigid linker amino acid sequence composed of glutamic acid (E), alanine (A), and lysine (K).

11. The recombinant KalbTGase substrate of claim 1, wherein at least one Lⁿ is selected from the group consisting of SEQ ID NO: 12, SEQ ID NO: 13, SEQ ID NO: 14, SEQ ID NO: 15, SEQ ID NO: 16, and SEQ ID NO: 17.

12. The recombinant KalbTGase substrate of claim 10, wherein at least one rigid linker amino acid sequence of T and/or any of R^{m} is an independently selected amino acid sequence of Formula IV
[E-[A-]ᵥK]_{w} (Formula IV),
wherein
v is an integer from 2 to 5, and
w is an integer from 1 to 6.

13. The recombinant KalbTGase substrate of claim 1, wherein at least one Lⁿ is selected from the group consisting of SEQ ID NO: 70, SEQ ID NO: 71, SEQ ID NO: 73, SEQ ID NO: 74, SEQ ID NO: 75, and SEQ ID NO: 76.

14. A method of forming a target polypeptide with a covalently attached label, the method comprising the steps of
(a) providing a KalbTGase substrate according to claim 1, the KalbTGase substrate comprising the target polypeptide and one or more acyl donor glutamine residue(s) for KalbTGase transglutaminase activity;
(b) providing a label conjugate, wherein in the label conjugate the label is covalently attached to a Ktag, wherein the Ktag is a KalbTG lysine-containing-acceptor-motif or a functional analog thereof, wherein the Ktag comprises a primary amine group capable of being reacted with an acyl donor glutamine residue for KalbTGase transglutaminase activity in the presence of KalbTGase; and
(c) contacting the fusion polypeptide of (a) and the conjugate of the label of (b) with KalbTGase under conditions allowing transglutaminase activity to occur;
thereby reacting an acyl donor glutamine residue with a primary amine group to form a covalent connection,
thereby forming the target polypeptide with the covalently attached label.

15. A method of forming a target polypeptide with a covalently attached capture group, the method comprising the steps of
(a) providing a KalbTGase substrate according to claim 1, the KalbTGase substrate comprising the target polypeptide and one or more acyl donor glutamine residue(s) for KalbTGase transglutaminase activity;
(b) providing a capture group conjugate, wherein in the capture group conjugate the capture group is covalently attached to a Ktag, wherein the Ktag is a KalbTG lysine-containing-acceptor-motif or a functional analog thereof, wherein the Ktag comprises a primary amine group capable of being reacted with an acyl donor glutamine residue for KalbTGase transglutaminase activity in the presence of KalbTGase; and
(c) contacting the fusion polypeptide of (a) and the conjugate of the capture group of (b) with KalbTGase under conditions allowing transglutaminase activity to occur,
thereby reacting an acyl donor glutamine residue with a primary amine group to form a covalent connection,
thereby forming the target polypeptide with the covalently attached capture group.

16. A labeled target polypeptide, obtained or obtainable by the method of claim 14.

17. A target polypeptide with a covalently attached capture group, obtained or obtainable by the method of claim 15.

18. A DNA encoding a fusion polypeptide comprising a target polypeptide and one or more acyl donor glutamine residue(s) for KalbTGase transglutaminase activity, wherein a codon for N-terminal methionine is appended to a nucleotide sequence encoding the amino acid sequence of the KalbTGase substrate of claim 1.

19. An expression vector for recombinant expression in a transformed organism, the expression vector comprising the DNA of claim 18.

20. A prokaryotic host organism stably transformed with an expression vector of claim 19, wherein the transformed host organism is capable of expressing the encoded KalbTGase substrate from the expression vector.

21. A method of producing a recombinant KalbTGase substrate, the method comprising the steps of
(a) providing and culturing the transformed host organism of claim 20;
(b) expressing in the transformed host organism of (a) the recombinant KalbTGase substrate, and
(c) purifying the expressed recombinant KalbTGase substrate from the transformed host organism,
thereby producing the recombinant KalbTGase substrate.

22. A composition suitable for detecting in an isolated sample target antibodies specific for an antigen amino acid sequence, wherein the composition comprises a labeled target polypeptide of claim 16, wherein the antigen amino acid sequence is comprised in the labeled target polypeptide.

23. The composition of claim 22, wherein the label is selected from the group consisting of a fluorescent dye, a chemiluminescent label, an iridium-containing electrochemiluminescent label, a ruthenium-containing electrochemiluminescent label, a single-stranded oligonucleotide or analog thereof, and a radiolabel.

24. The composition of claim 22, wherein the composition further comprises a separate molecule comprising the unlabeled antigen amino acid sequence or a molecular mimic thereof attached to a capture group.

25. The composition of claim 24, wherein the separate molecule is a target polypeptide with a covalently attached capture group.

26. A method for detecting in an isolated sample a target antibody (═X) specific for an antigen amino acid sequence (═Y), said method comprising
(a) forming an immunoreaction mixture by admixing a body fluid sample suspected of containing X with a labeled target polypeptide of claim 16, wherein Y is comprised in the labeled target polypeptide;
(b) maintaining the immunoreaction admixture of step (a) for a time period sufficient for allowing X present in the body fluid sample to immunoreact with Y comprised in the labeled target polypeptide to form an immunoreaction product (═X:Y) in the admixture; and
(c) detecting the presence and/or the concentration of X:Y formed in step (b),
thereby detecting in the isolated sample target antibody specific for the antigen amino acid sequence.

27. The method of claim 26, wherein step (c) comprises the steps of
(i) contacting the immunoreaction admixture of step (a) or the admixture with immunoreaction product of step (b) with a separate molecule (═Z), wherein Z comprises a capture group and the unlabeled antigen amino acid sequence or a molecular mimic thereof,
(ii) maintaining the admixture of step (i) for a time period sufficient for allowing an immunoreaction product to form in which X simultaneously binds Y and Z (═Z:X:Y);
(iii) capturing Z:X:Y and separating Z:X:Y from the admixture of step (ii); and
(iv) detecting the label present in captured Z:X:Y.

28. The method of claim 27, wherein Z is a target polypeptide with a covalently attached capture group.

29. The method of claim 27, wherein step (iii) comprises capturing Z:X:Y on a solid phase, and separating the solid phase with captured Z:X:Y from the admixture of step (ii).

30. (canceled)

31. (canceled)

32. A kit of parts for detecting in an isolated sample target antibodies specific for an antigen amino acid sequence, the kit containing a labeled target polypeptide of claim 16, the labeled target polypeptide comprising the antigen amino acid sequence.
